(19) United States
(12) Patent Application Publication
(10) Pub. No.: US 2008/0305986 A1
(43) Pub. Date: Dec. 11, 2008

(54) Multimers of Peptides

(76) Inventors: Killian Waldemar Conde Fieboes, Malov (DK); Birgitte Urs, Copenhagen (DK); Lars Fogh Iversen, Holte (DK)

Correspondence Address: NOVO NORDISK, INC.;INTELLECTUAL PROPERTY DEPARTMENT, 100 COLLEGE ROAD WEST, PRINCETON, NJ, 08540, US

(21) Appl. No.: 11/660,398

(22) PCT Filed: Aug. 16, 2005
(86) PCT No.: PCT/EP05/54020

§ 371 Date: Sep. 20, 2007

(30) Foreign Application Priority Data
Aug. 16, 2004 (DK) PA 2004 01232
Sep. 27, 2004 (DK) PA 2004 01466
Apr. 14, 2005 (DK) PA 2005 00539

(51) Int. Cl. A61K 38/02; C07C 237/04; C07D 251/30; C07D 207/40; C07K 14/705; C07K 14/62; C07K 14/61; C07H 21/00

(52) U.S. Cl. 514/2; 564/153; 544/222; 548/546; 530/300; 530/303; 530/350; 536/23.1

(57) ABSTRACT

Described are compounds useful for trimerising chemical entities, methods of trimerising chemical entities, and trimerised entities. In one aspect, the entities are peptides.

### FIELD OF THE INVENTION

The present invention relates to a novel trimerisation molecule, methods of trimerising peptides via the novel chemical entity, as well as trimerised peptide molecules.

### BACKGROUND OF THE INVENTION

Cytokines are involved in regulations of the immune system through receptor molecules, stimulating signal transduction leading to modulation of cellular secretion, growth and mobility. They act in a local or systemic manner, regulating a variety of biological processes such as immunity, inflammation and hematopoiesis. They are produced by a variety of cells including fibroblasts, macrophages and lymphocytes. Cytokines differ from hormones in that they are secreted from a variety of cells rather than a specific organ. A large number of cytokines have been identified.

Modulation of cytokine can be used as therapeutic intervention. This can be achieved by, e.g., soluble receptor molecules sequestering the cytokine or antibodies blocking the cytokine or its ligand. One such cytokine, TNFα (a 17 kDa monomer) and the 17 members of its family are trimeric ligands. These presently known 17 trimeric ligands of the TNF family are: LTA, TNF, LTB, OX40L, CD40L, FasL, CD27L, CD30L, 4-IBB-L, TRAIL, RANKL, TWEAK, APRIL, BAFF, LIGHT, VEGI and GITRL.

Evidence that these trimeric ligands bind to trimeric receptors have been published (Bodmer, TRENDS in Biochem Sc., Vol. 27, pp. 19-26 (2002)). For TNFα two receptors exist, the TNFR1 of 55 kDa and TNFR2 of 75 kDa. Both receptors are non-covalently linked trimers on the cell surface interacting via PLAD (pre ligand assembly domain, Chan F K et al., Science, Vol. 288 pp. 2351-2354 (2000)). Other members of the TNFR super family also posses PLAD's, including TRAIL receptor1, CD40, Fas LTRbetaR, CD30, CD27, HVEMm, RANK OX40 and DR4. Other members include the soluble receptors (s-TNFR) of different sizes. The 55 KDa and 75 KDa TNFR peptides, as well as shorter fragments, have been described in EP422339 and EP556207.

EP526905 describes multimerisation of soluble forms of TNF receptors by adding a C-terminal cysteine or biotin group to monomers.

WO9952877 and WO2003102207 describe multimerization of receptor ligands using specific molecules.

Halazy (Exp Opin Ther Patents 1999; 9:431-446) discusses the design of bivalent ligands for G-protein coupled receptors.

The present invention provides methods and trimerisation molecules suitable for trimerisation of any peptide.

### SUMMARY OF THE INVENTION

The invention provides methods and trimerisation molecules suitable for conjugating peptides. The invention also provides the conjugate of peptides trimerised by this trimerisation molecule.

The trimerisation molecules provided are of the general formula

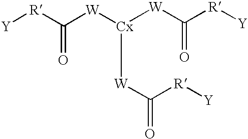

wherein Cx represents a center moiety, which is supporting three independent arms; and

each W independently represents the di-radicals of C₁₋₆-alkylene, C₁₋₆-alkeneoxy-C₁₋₆-alkylene, C₂₋₆-alkenylene, C₂₋₆-alkynylene, hydroxy-C₁₋₆-alkylene, hydroxy-C₂₋₆-alkenylene, C₁₋₆-alkanoylene, C₂₋₆-alkenoylene or a bond,

each R′ is the same or different, and comprises one or more of the group A and optionally one or more of the group B, each R′ starting with A,

wherein A is

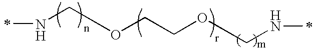

wherein r is any number from 1-50

n and m are integers ≧2; and

B is

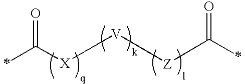

wherein X, V and Z are independently selected from —CR¹R²—, C₃₋₈-cycloalkylene, C₄₋₈-cycloalkenylene, -arylene-, -heteroarylene-, -heterocyclylene-, —O—, —S—, NR¹, —OCH₂CH₂O—, —OCH₂—, —CH₂O—, and

R¹ and R² are independently selected from H, C₁₋₆-alkyl, C₂₋₆-alkenyl, C₃₋₈-cycloalkyl, C₄₋₈-cycloalkenyl, or R¹ and R² can be taken together to form a C₂₋₆-alkylene-bridge;

q, k and l are independently selected from 0, 1, 2, 3, 4, 5 or 6, but they are not all simulataneously 0; and

Y is a group suitable for covalent attachment to a peptide.

The invention also provides peptide trimers, where the molecules described above have been conjugated to peptides as shown below, wherein each group Y has been reacted with a peptide P to form Y′P′.

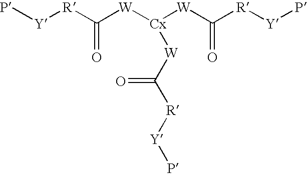

The invention also provides pharmaceutical compositions comprising the peptide trimer conjugates.

The invention also provides the use of these peptide trimer conjugates for the treatment of diseases.

The invention also provides methods of trimerising peptides or other chemical entities using the trimerisation molecules described herein.

The invention also provides peptide sequences particular useful for conjugation to these trimerisation molecules and the corresponding nucleic acids for expression in host cells.

In an aspect of the invention, a trimerised peptide using the trimerisation molecules provided by the present invention has improved affinity of the trimerised peptide as compared to the native peptide which is not trimerised.

In an aspect of the invention, a trimerised peptide using the trimerisation molecules provided by the present invention has improved efficacy as compared to the native peptide.

In an aspect of the invention, a trimerised peptide using the trimerisation molecules provided in the present invention has improved functional in vivo half life as compared to the native peptide.

In an aspect of the invention, a trimerised peptide using the trimerisation molecules provided by the present invention has improved physical stability as compared to the native peptide.

### DEFINITIONS

The terms “C₁₋₆-alkyl” or “C₁₋₆-alkylene” refer to a saturated, branched or straight hydrocarbon group having from 1 to 6 carbon atoms. Typical C₁₋₆-alkyl groups include, but are not limited to, methyl, ethyl, n-propyl, isopropyl, butyl, isobutyl, sec-butyl, tert-butyl, pentyl, hexyl and the corresponding divalent radicals.

The terms “C₂₋₆-alkenyl” or “C₂₋₆-alkenylene” refer to a branched or straight hydrocarbon group having from 2 to 6 carbon atoms and at least one double bond. Typical C₂₋₆-alkenyl groups include, but are not limited to, ethenyl, 1-propenyl, 2-propenyl, isopropenyl, 1,3-butadienyl, 1-butenyl, 2-butenyl, 1-pentenyl, 2-pentenyl, 1-hexenyl, 2-hexenyl, 1-ethylprop-2-enyl, 1,1-(dimethyl)prop-2-enyl, 1-ethylbut-3-enyl, 1,1-(dimethyl)but-2-enyl, and the corresponding divalent radicals.

The terms “C₂₋₆-alkynyl” or “C₂₋₆-alkynylene” refer to a branched or straight hydrocarbon group having from 2 to 6 carbon atoms and at least one triple bond. Typical C₂₋₆-alkynyl groups include, but are not limited to, vinyl, 1-propynyl, 2-propynyl, isopropynyl, 1,3-butadynyl, 1-butynyl, 2-butynyl, 1-pentynyl, 2-pentynyl, 1-hexynyl, 2-hexynyl, 1-ethylprop-2-ynyl, 1,1-(dimethyl)prop-2-ynyl, 1-ethylbut-3-ynyl, 1,1-(dimethyl)but-2-ynyl, and the corresponding divalent radicals.

The terms “C₁₋₆-alkyloxy”, “C₁₋₆-alkyleneoxy”, “C₂₋₆-alkenyloxy”, “C₂₋₆-alkenyleneoxy”, “C₂₋₆-alkynyloxy” or “C₂₋₆-alkynyleneoxy”; refer to the radical —O—C₁₋₆-alkyl, —O—C₁₋₆-alkylene, —O—C₂₋₆-alkenyl, —O—C₂₋₆-alkenylene, —O—C₂₋₆-alkynyl or —O—C₂₋₆-alkynylene wherein C₁₋₆-alkyl(ene), C₂₋₆-alkenyl or C₂₋₆-alkynyl are as defined above. Representative examples are methoxy, ethoxy, n-propoxy, isopropoxy, butoxy, sec-butoxy, tert-butoxy, pentoxy, isopentoxy, hexoxy, isohexoxy and the like.

The terms “C₁₋₆-alkylthio”, “C₁₋₆-alkylenethio”, “C₂₋₆-alkenylthio”, “C₂₋₆-alkenylenethio”, “C₂₋₆-alkynylthio” or “C₂₋₆-alkynylenethio”; refer to the corresponding thio analogues of the oxy-radicals as defined above. Representative examples are methylthio, ethylthio, propylthio, butylthio, pentylthio, hexylthio, and the corresponding divalent radicals and the corresponding alkenyl and alkynyl derivatives also defined above.

In the context of this invention the term “-triyl” is used and refers to different alkyl, alkenyl, alkynyl, cycloalkyl or aromatic radicals with three attachment points.

The terms “C₃₋₈-cycloalkyl” or “C₃₋₈-cycloalkylene” refer to a monocyclic, carbocyclic group having from 3 to 8 carbon atoms or the corresponding biradical. Representative examples are cyclopropyl, cyclobutyl, cyclopentyl, cyclohexyl, cycloheptyl, cyclooctyl, and the like.

The terms “C₄₋₈-cycloalkenyl” or “C₄₋₈-cycloalkenylene” refer to C₄₋₈-cycloalkenyl representing a monocyclic, carbocyclic, non-aromatic group having from 4 to 8 carbon atoms or the corresponding biradical and at least one double bond. Representative examples are cyclobutenyl, cyclopentenyl, cyclohexenyl, cycloheptenyl, cyclooctenyl and the like.

The terms “aryl” or “arylene” as used herein are intended to include carbocyclic aromatic ring systems such as phenyl, biphenylyl, naphthyl, anthracenyl, phenanthrenyl, fluorenyl, indenyl, pentalenyl, azulenyl and the like and the corresponding biradicals. Aryl or Arylene are also intended to include the partially hydrogenated derivatives of the carbocyclic systems. Non-limiting examples of such partially hydrogenated derivatives are 1,2,3,4-tetrahydronaphthyl, 1,4-dihydronaphthyl and the like.

The terms “heteroaryl” or “heteroarylene” as used herein are intended to include heterocyclic aromatic ring systems containing one or more heteroatoms selected from nitrogen, oxygen and sulfur such as furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5-triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, benzothiophenyl (thianaphthenyl), indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like and the corresponding biradicals. Heteroaryl or heteroarylene are also intended to include the partially hydrogenated derivatives of the heterocyclic systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

The terms “heterocyclyl” or “hetereocycylene as used herein denote the fully hydrogenated derivatives of the heteroaryls or the heteroarylenes, respectively, defined above.

The term heteroaryl-C₁₋₆-alkyl as used herein denotes heteroaryl as defined above and C₁₋₆-alkyl as defined above.

The terms “aryl-C₁₋₆-alkyl” and “aryl-C₂₋₆-alkenyl” as used herein denote aryl as defined above and C₁₋₆-alkyl and C₂₋₆-alkenyl, respectively, as defined above.

The term “acyl” as used herein denotes —(C═O)—C₁₋₆-alkyl wherein C₁₋₆-alkyl is as defined above.

The terms “C₂₋₆-alkanoyl” or “C₂₋₆-alkanoylene” as used herein denote —(C═O)—C₁₋₅-alkyl or —(C═O)—C₁₋₅-alkylene, respectively.

The terms “C₃₋₆-alkenoyl” or “C₃₋₆-alkenoylene” as used herein denote —(C═O)—C₂₋₅-alkenyl or —(C═O)—C₂₋₅-alkenylene, respectively.

The terms “peptide”, “polypeptide”, “oligopeptide” or protein may be interchanged and used in the context of the present invention without the intention to distinguish on the basis of length or weight but only indicate that amino acids are covalently attached to each other.

The term “optionally substituted” as used herein means that the groups in question are either unsubstituted or substituted with one or more of the substituents specified. When the groups in question are substituted with more than one substituent, the substituents may be the same or different.

The terms “treatment” and “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive therapy, whereas “treatment” of a patient in whom symptoms or clinically relevant manifestations of a disease or disorder have been identified generally does not constitute preventive therapy. Nonetheless, it should be understood that the various therapeutic and prophylactic methods of the invention are distinct in many respects (e.g., dosages, administration schedule, etc.) and may each be considered a unique aspect of the invention.

Certain of the above defined terms may occur more than once in the structural formulae, and upon such occurrence each term shall be defined independently of the other.

Furthermore, when using the terms “independently are” and “independently selected from” it should be understood that the groups in question may be the same or different.

### DETAILED DESCRIPTION OF THE INVENTION

The present invention provides methods of trimerising peptides, compounds useful as trimerisation molecules for any peptide, and trimerised peptides.

The center moiety Cx can be any suitable molecule, including, but not limited to, tri-radicals of cyclohexane, benzene, nitrogen and [1,3,5]Triazinane-2,4,6-trione. In an aspect of the invention, Cx is a tri-radical of nitrogen. In another aspect, Cx is a tri-radical of cyclohexane. In another aspect, Cx is a tri-radical of benzene. In another aspect. Cx is a tri-radical of 1,3,5]Triazinane-2,4,6-trione.

In an aspect of the invention, the trimerisation molecule as shown by the general formula above are compounds having any of the formulas below:

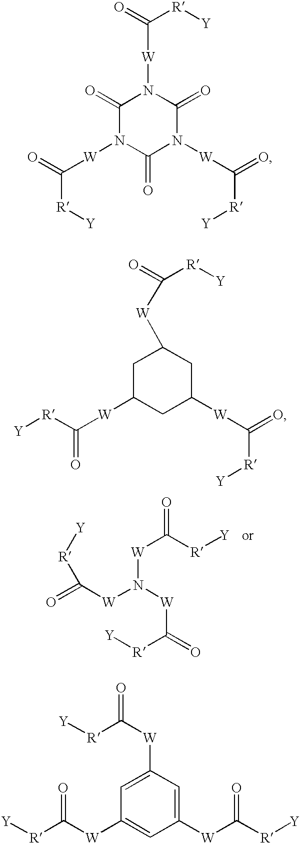

wherein R′, W and Y are as defined above. In an aspect of the invention, W is a bond or C₁₋₆-alkylene.

In an aspect of the invention the trimerisation molecules are more specifically represented by any of the formulas below:

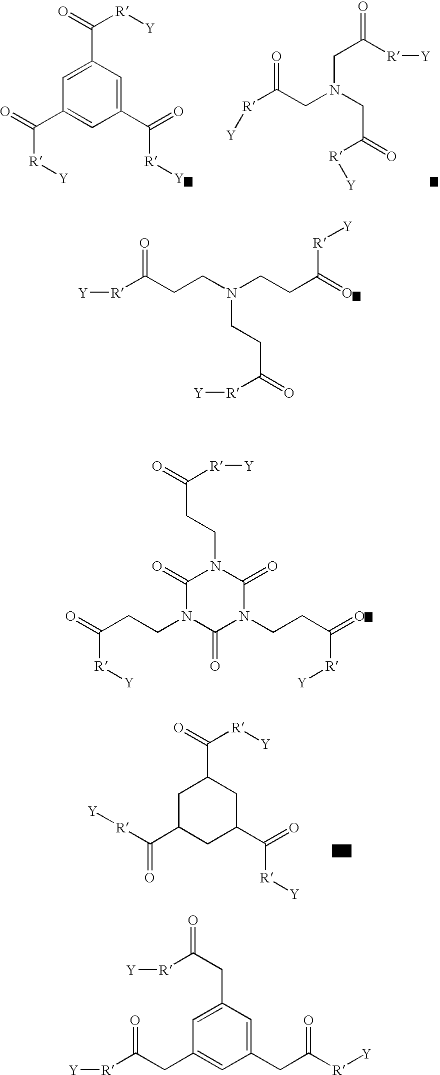

wherein R′ and Y are is as defined above.

In an aspect of the invention, A is represented by the formula below

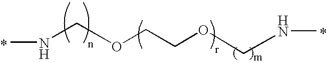

wherein r is 0-25; and n and m are as defined above. In an aspect of the invention, r is 0-10. In an aspect of the invention, r is 0-5. In an aspect of the invention, r is 0, 1, 2 or 3.

Each R′ may be the same or different. In one aspect, all R′ are the same molecule. In one aspect, two R′ are the same molecule. In one aspect, all R′ are different molecules.

In an aspect of the invention n and m are below 15. In an aspect of the invention n and m are below 10. In an aspect of the invention n and m are below 6. In an aspect of the invention n and m are independently selected from 2, 3, 4 or 5. In an aspect of the invention n and m are independently selected from 2 or 3.

In an aspect of the invention, A is any of the structures below:

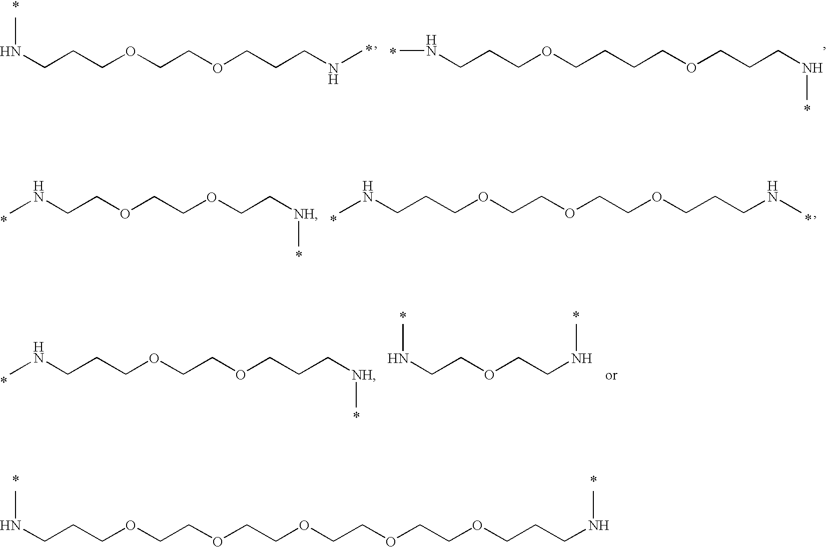

In an aspect of the invention, the element B as defined in the formula above, and X, V and Z independently represent —CR¹R²—, —O—, —S—, NR¹, —OCH₂CH₂O—, —OCH₂— or —CH₂O—

In an aspect of the invention, X, V and Z represent —CR¹R²— wherein R¹ and R² are as defined above.

In an aspect of the invention, X, V and Z independently represent —OCH₂CH₂O—, —OCH₂— or —CH₂O—.

In an aspect of the invention, X, V and Z independently represent —(CH₂)₁₋₄—, —CH₂(OCH₂CH₂O)₁₋₆CH₂— or —CH₂OCH₂—.

In an aspect of the invention, X, V and Z independently represent —(CH₂)₃— or —CH₂OCH₂—.

In an aspect of the invention, B is any of the structures below:

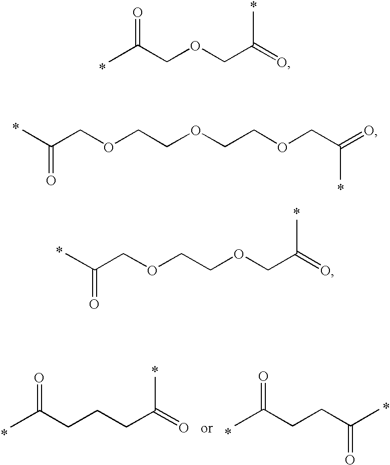

In an aspect of the invention, X, V and Z independently represent aryl or heteroaryl.

In an aspect of the invention, V is C₃₋₈-cycloalkyl or C₃₋₈-cycloalkenyl.

In an aspect of the invention, V is cyclohexyl, cyclohexenyl, cyclopentyl.

In an aspect of the invention, V is phenyl.

In an aspect of the invention, V is morpholinyl, piperazinyl, dioxanyl, or thienyl.

The group Y″, which can be defined as the monomeric building block leading to Y in the formula above and to Y′ when attached to a peptide, has two reactive functional groups.

It reacts with either group A or group B depending on the characteristics of the R′ when assembling the trimerisation molecule. Thus, if R′ has been chosen to contain a B as the last constituent of the R′, Y″ should contain a reactive functional group that can be attached to an acid derivate. If the R′ has been chosen to contain an A group as the last constituent of the R′, Y″ should contain a reactive functional group suitable for reacting with amines. The other functionality of Y″ and Y should be able to react with peptides. This could be side chains of, for example, the natural occurring peptides of which some contains functional groups, or compounds attached to these aminoacids either internally in the sequence, or in the N- or C-terminal.

If suitable attachment groups such as amines, thiols or hydroxyl groups are not already present on the peptide, or if modification of these interfere with the biological function of the peptide, suitable attachment groups are created on the native peptide by conventional genetic engineering such as, e.g., mutation on the DNA—level (e.g. coding codon replacement) of selected amino acids with amino acids allowing for post modificational attachment of polymers. The choice of which amino acid to mutate depend on the particular peptide. In general, it is desirable to select “allowed mutations”, e.g., to select mutations in amino acids that will not affect the binding of the peptide to its natural ligands, or inhibit the peptides biological function such as enzymatic actions, substrate binding, etc. Such pre- or post-translationally modified peptides are examples of analogues of the original peptides.

Mutation of DNA sequences using nonsense amber (stop) codons in conjunction with new genetically mutated tRNA synthetases, selected to accept unnatural amino acids, is also a way to prepare peptides with unnatural amino acids under in vivo fermentation conditions (Wang, L. et al. PNAS U.S.A., 2003, 100, 56-61). Additionally, incorporation of novel amino acids with unique functional attachment groups, and post modification of these with glycomimetics is demonstrated (Liu, H.; Wang, L.; Brock, A.; Wong, C.-H.; Schultz, P. G.; J. Am. Chem. Soc.; (Communication); 2003; 125; 1702-1703). These gene products are suitable peptides according to the invention, as new non-natural chemoselective attachment moieties become available for modification with branched polymers.

In an aspect of the invention the peptide is assembled on solid phase and selected amino acids are substituted with amino acids with suitable side chains acting as attachment groups, using standard solid phase chemistry. Examples of such amino acid substitutions are by way of non-limiting illustration: substitution of serine with cystein, substitution of phenylalanine with tyrosine, or substitution of arginine with lysine. Alternatively, attachment groups are introduced by enzyme directed coupling in either the C- or N-terminal end of the peptide, with either suitable amino acids allowing for post modificational attachment of polymers, or small organic molecules serving the same purpose. Enzymes that support this aspect of the invention include by way of non-limiting illustration: carboxypeptidases, and proteases in reverse.

The term “reactive functional group” means by way of illustration and not limitation, any free amino, carboxyl, thiol, alkyl halide, acyl halide, chloroformate, aryloxycarbonate, hydroxy or aldehyde group, carbonates such as the p-nitrophenyl, or succinimidyl; carbonyl imidazoles, carbonyl chlorides; carboxylic acids that are activated in situ; carbonyl halides, activated esters such as N-hydroxysuccinimide esters, N-hydroxybenzotriazole esters, esters of such as those comprising 1,2,3-benzotriazin-4(3″-one, phosphoramidites and H-phosphonates, phosphortriesters or phosphordiesters activates in situ, isocyanates or isothiocyanates, in addition to groups such as NH₂, OH, N₃, NHR′, OR′, O—NH₂, alkynes, or any of the following

| | |
|---|---|
| hydrazine derivatives | —NH—NH₂, |
| hydrazine carboxylate | —O—C(O)—NH—NH₂, |
| derivatives | |
| semicarbazide derivatives | —NH—C(O)—NH—NH₂, |
| thiosemicarbazide | —NH—C(S)—NH—NH₂, |
| derivatives | |
| carbonic acid dihydrazide | —NHC(O)—NH—NH—C(O)—NH—NH₂, |
| derivatives | |
| carbazide derivatives | —NH—NH—C(O)—NH—NH₂, |
| thiocarbazide derivatives | —NH—NH—C(S)—NH—NH₂, |
| aryl hydrazine | —NH—C(O)—C₆H₄—NH—NH₂, |
| derivatives | |
| hydrazide derivatives | —C(O)—NH—NH₂; and |
| oxylamine derivatives | C(O)—O—NH₂, —NH—C(O)—O—NH₂ and |
| | —NH—C(S)—O—NH₂. |

Other functional groups present may be suitably protected by protection groups. Appropriate protection groups are known to the skilled person, and examples can be found in, e.g., Green & Wuts “Protection groups in organic synthesis”, 3.ed. Wiley-interscience.

Thus in an aspect of the invention, Y is represented by the following structures:

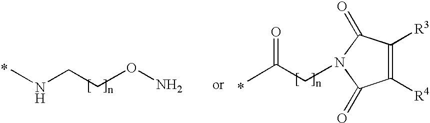

wherein n is an integer ≧0, and R³ and R⁴ independently represents hydrogen, or C₁₋₆-alkyl.

In an aspect of the invention, Y is

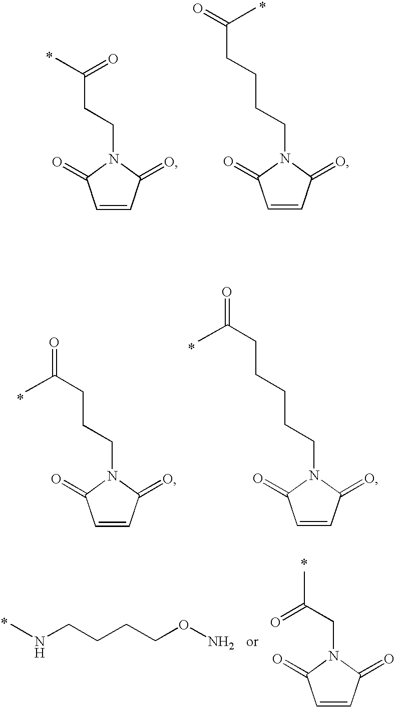

wherein n, R³ and R⁴ are as defined above.

In an aspect of the invention, Y is represented by

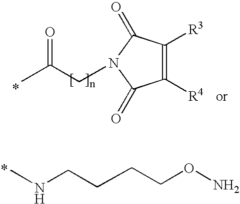

wherein R³, R⁴ and n are as defined above.

In an aspect of the invention, R′ is selected from one of the following combinations: A-B, A-B-A-B, A-B-A-B-A-B, A, A-B-A or A-B-A-B-A.

In an aspect of the invention, all R's are identical

In an aspect of the invention, two R′ are identical

In an aspect of the invention, R′ are different.

According to the present invention, the trimerisation molecules are assembled according to the methods described. The peptides are then coupled.

Trimerisation molecules having a maleimide moiety can be coupled to peptides via a Michael reaction with a free cysteine on the peptide.

Trimerisation molecules having an aminooxy moiety can be coupled to peptides via the formation of an oxime with a keto or aldehyde group on the peptide.

The present invention provides compounds useful as trimerisation molecules for any peptide. Some peptides can act in their natural environment as a trimer. This includes, for example, TNFα. TNFα is involved in the pathophysiology of rheumatoid arthritis, an inflammatory disease that affects approximately 1% of the population. Inhibition of TNFα by soluble receptors or antibodies are currently used for treatment of the disease. Furthermore, TNFα is indicated in a number of other inflammatory diseases.

In an aspect of the invention, the peptide conjugated to the trimerisation molecule is a member of the TNFR superfamily. In another aspect, the peptide is a fragment or a functional analogue of a fragment of a member of the TNFR superfamily. Exemplary functional analogues include those with N- and/or C-terminal mutations, derivatizations, or substitutions to facilitate trimerisation or other post-translational modifications.

In an aspect of the invention, the peptide is a functional analogue of a fragment of a TNF receptor molecule, the peptide consisting of or comprising any one of the following sequences:

(SEQ ID NO:1)

DSVCPQGKYIHPQNNSICCTKCHKGTYLYNDCPGPGQDTDCRECESGSFT
ASENHLRHCLSCSKCRKEMGQVEISSCTVDRDTVCGCRKNQYRHYWSENL
FQCFNCSLCLNGTVHLSCQEKQNTVCTSHAA
and

(SEQ ID NO:2)

DSVCPQGKYIHPQNNSICCTKCHKGTYLYNDCPGPGQDTDCRECESGSFT
ASENHLRHCLSCSKCRKEMGQVEISSCTVDRDTVCGCRKNQYRHYWSENL
FQCFNCSLCLNGTVHLSCQEKQNTVCTSHAC.

In an aspect of the invention, the peptide is a functional analogue of a fragment of a TNF receptor molecule, the peptide consisting of or comprising any one of the following sequences:

(SEQ ID NO:3)

SVCPQGKYIHPQNNSICCTKCHKGTYLYNDCPGPGQDTDCRECESGSFTA
SENHLRHCLSCSKCRKEMGQVEISSCTVDRDTVCGCRKNQYRHYWSENLF
QCFNCSLCLNGTVHLSCQEKQNTVCTLLA

(SEQ ID NO:4)

SVCPQGKYIHPQNNSICCTKCHKGTYLYNDCPGPGQDTDCRECESGSFTA
SENHLRHCLSCSKCRKEMGQVEISSCTVDRDTVCGCRKNQYRHYWSENLF
QCFNCSLCLNGTVHLSCQEKQNTVCTSHALLA

(SEQ ID NO:5)

SVCPQGKYIHPQNNSICCTKCHKGTYLYNDCPGPGQDTDCRECESGSFTA
SENHLRHCLSCSKCRKEMGQVEISSCTVDRDTVCGCRKNQYRHYWSENLF
QCFNCSLCLNGTVHLSCQEKQNTVC

(SEQ ID NO:6)

DSVCPQGKYIHPQNNSICCTKCHKGTYLYNDCPGPGQDTDCRECESGSFT
ASENHLRHCLSCSKCRKEMGQVEISSCTVDRDTVCGCRKNQYRHYWSENL
FQCFNCSLCLNGTVHLSCQEKQNTVCTLLA
and

(SEQ ID NO:7)

DSVCPQGKYIHPQNNSICCTKCHKGTYLYNDCPGPGQDTDCRECESGSFT
ASENHLRHCLSCSKCRKEMGQVEISSCTVDRDTVCGCRKNQYRHYWSENL
FQCFNCSLCLNGTVHLSCQEKQNTVCTSHALLA.

In an aspect of the invention, one or more of SEQ ID NOS:1-7 further comprise an N-terminal methionine (M) residue, representing the start codon. In one particular aspect, when expressed in vivo, this residue is cleaved off.

Currently used inhibitors of TNFα, used for, e.g., rheumatoid arthritis patients include etanercept (a TNFR 75-IgG1 hinge and Fc domain fusion protein), infliximab (a chimeric anti-TNFα antibody made of variable regions from mouse and all other domains from human IgG1) and adalimumab (a fully human anti-TNFα IgG1 antibody). These inhibitors are dimeric molecules, possibly leaving one binding site of the ligand uninhibited.

In practising the invention, the affinity of the trimer for a target can be higher than the affinity of the dimer or the monomer. The trimerisation can also lead to higher efficacy. The functional in vivo half life of the molecule might be altered due to its altered size, resulting in, e.g. exclusion from glomerular filtration, or due to altered biodistribution. In an embodiment of the invention, the in vivo half-life is increased. Also, the stability or physical half life might be changed due to its trimeric state. As an example, the trimerised TNFR is not secreted through the kidney and is a molecule with increased affinity as compared to monomeric TNFR.

The term “functional in vivo half-life” is used in its normal meaning, i.e., the time at which 50% of the biological activity of the polypeptide or conjugate is still present in the body/target organ, or the time at which the activity of the polypeptide or conjugate is 50% of its initial value. As an alternative to determining functional in vivo half-life, “serum half-life” may be determined, i.e., the time at which 50% of the polypeptide or conjugate molecules circulate in the plasma or bloodstream prior to being cleared. Determination of serum-half-life is often more simple than determining functional half-life and the magnitude of serum-half-life is usually a good indication of the magnitude of functional in vivo half-life. Alternative terms to serum half-life include plasma half-life, circulating half-life, circulatory half-life, serum clearance, plasma clearance, and clearance half-life. The functionality to be retained is normally selected from procoagulant, proteolytic, co-factor binding, receptor binding activity, or other type of biological activity associated with the particular peptide.

The term “increased” as used about the functional in vivo half-life or plasma half-life is used to indicate that the relevant half-life of the polypeptide or conjugate is statistically significantly increased relative to that of a reference molecule, such as non-conjugated glycopeptide as determined under comparable conditions. For instance the relevant half-life may be increased by at least about 25%, such as by at least about 50%, e.g., by at least about 100%, 150%, 200%, 250%, or 500%.

In an aspect of the invention other polypeptides which functions as a trimer in their physiological setting can be trimerised in the described way, non limiting examples are members of the TNFR super family TRAIL receptor1, CD40, Fas LTRbetaR, CD30, CD27, HVEMm, RANK, OX40 and DR4. Furthermore, other polypeptides not normally found in a biological trimeric setting can also be trimerised, changing some of the above described parameters. For example, as described elsewhere herein, trimerisation can prolong the functional in vivo half-life of a peptide and/or increase the concentration of ligand near its receptor. Not limiting examples of polypeptides are albumin, Fab fragments of antibodies, enzymes, peptide hormones, growth factors, antibodies, peptides binding to the melanocortin receptor class (MCR1-5), cytokines, receptors, lymphokines and vaccine antigenes, and particular mentioning is made of therapeutic peptides, such as TNF, insulin, glucagon like-peptide 1 (GLP-1), glucagon like-peptide 2 (GLP-2), growth hormones such as, e.g., human growth hormone (hGH); cytokines, trefoil factor peptides (TFF), peptide melanocortin receptor modifiers and factor VII.

Any peptide can be conjugated to the molecules of the present invention by the methods described, such as, e.g., enzymes, peptide hormones, growth factors, antibodies, cytokines, receptors, lymphokines and vaccine antigenes, and particular mentioning is made of therapeutic peptides, such as TNF, insulin, glucagon like-peptide 1 (GLP-1), glucagon like-peptide 2 (GLP-2), growth hormone, cytokines, trefoil factor peptides (TFF), peptide melanocortin receptor modifiers and factor VII compounds.

Particular applicable insulin is human insulin and functional analogues thereof. In the present context the term “human insulin” refers to naturally produced insulin or recombinantly produced insulin. Recombinant human insulin may be produced in any suitable host cell, for example the host cells may be bacterial, fungal (including yeast), insect, animal or plant cells. Many insulin compounds have been disclosed in the literature, and they too are particular useful in the methods of the present invention. By “insulin compound” (and related expressions) is meant human insulin in which one or more amino acids have been deleted and/or replaced by other amino acids, including non-codeable amino acids, and/or human insulin comprising additional amino acids, i.e. more than 51 amino acids, and/or human insulin in which at least one organic substituent is bound to one or more of the amino acids.

In different embodiments of the invention, the peptide is aprotinin, tissue factor pathway inhibitor or other protease inhibitors, insulin or insulin precursors, human or bovine growth hormone, interleukin, glucagon, Glucagon(1-37) (oxyntomodulin), GLP-1, GLP-2, IGF-1, IGF-II, tissue plasminogen activator, transforming growth factor α or β, platelet-derived growth factor, GRF (growth hormone releasing factor), immunoglubolines, EPO, TPA, protein C, blood coagulation factors such as FVII, FVIII, FIV and FXIII, exendin-3, exentidin-4, A-MSH, and enzymes or functional analogues thereof.

In the present context, the term “functional analogue” is meant to indicate a peptide with a similar function as the native peptide. The peptide may be structurally similar to the native peptide and may be derived from the native peptide by addition of one or more amino acids to either or both the C- and N-terminal end of the native peptide, substitution of one or more amino acids at one or a number of different sites in the native amino acid sequence, deletion of one or more amino acids at either or both ends of the native peptide or at one or several sites in the amino acid sequence, or insertion of one or more amino acids at one or more sites in the native amino acid sequence. Furthermore the peptide may be acylated in one or more positions, as described in, e.g., WO 98/08871 which discloses acylation of GLP-1 and analogues thereof, and in WO 98/08872 which discloses acylation of GLP-2 and analogues thereof. An example of an acylated GLP-1 derivative is Lys²⁶(N^{ε}-tetradecanoyl)-GLP-1(7-37) which is GLP-1(7-37) wherein the amino group of the Lys residue in position 26 has been tetradecanoylated.

Some peptides such as the interleukins, interferons and colony stimulating factors also exist in non-glycosylated form, usually as a result of using recombinant techniques. The non-glycosylated versions are also among the biologically active peptides of the present invention. The biologically active peptides of the present invention also include any portion of a peptide demonstrating in vivo bioactivity. This includes amino acid sequences, antibody fragments, single chain binding antigens, (see, for example U.S. Pat. No. 4,946,778), and binding molecules such as fusions of antibodies or fragments, polyclonal antibodies, monoclonal antibodies, and catalytic antibodies.

Non-limiting examples of other peptides or peptides applicable in the methods of the present invention include ACTH, corticotropin-releasing factor, angiotensin, calcitonin, insulin and fragments and analogues thereof, glucagon, IGF-1, IGF-2, enterogastrin, gastrin, tetragastrin, pentagastrin, urogastrin, epidermal growth factor, secretin, nerve growth factor, thyrotropin releasing hormone, somatostatin, growth hormone releasing hormone, somatomedin, parathyroid hormone, thrombopoietin, erythropoietin, hypothalamic releasing factors, prolactin, thyroid stimulating hormones, endorphins, enkephalins, vasopressin, oxytocin, opiods and analogues thereof, asparaginase, arginase, arginine deaminase, adenosine deaminase and ribonuclease and functional analogues of each of the mentioned peptides.

In an aspect of the invention, the full extracellular domain of TNFR 1 has the sequence:

(SEQ ID NO:8)

DSVCPQGKYIHPQNNSICCTKCHKGTYLYNDCPGPGQDTDCRECESGSFT
ASENHLRHCLSCSKCRKEMGQVEISSCTVDRDTVCGCRKNQYRHYWSENL
FQCFNCSLCLNGTVHLSCQEKQNTVCTCHAGFFLRENECVSCSNCKKSLE
CTKLCLPQIENVKGTEDSGTT

or the sequence of the naturally occurring circulating form of the above, as disclosed in EP422339:

(SEQ ID NO:9)

DSVCPQGKYIHPQNNSICCTKCHKGTYLYNDCPGPGQDTDCRECESGSFT
ASENHLRHCLSCSKCRKEMGQVEISSCTVDRDTVCGCRKNQYRHYWSENL
FQCFNCSLCLNGTVHLSCQEKQNTVCTCHAGFFLRENECVSCSNCKKSLE
CTKLCLPQIEN

TNFR 2 (as disclosed for example in EP422339) can also be used for trimerisation on the trimerisation molecule with the malimide as connection to the peptide.

Alternatively, a fraction of the TNFR1 55 kDa sequence, down to the minimal TNF binding fragment, can be used, such as the following, as mentioned in EP556207:

(SEQ ID NO:10)

DSVCPQGKYIHPQNNSICCTKCHKGTYLYNDCPGPGQDTDCRECESGSFT
ASENHLRHCLSCSKCRKEMGQVEISSCTVDRDTVCGCRKNQYRHYWSENL
FQCFNCSLCLNGTVHLSCQEKQNTVCTC.

Optionally, the sequences mentioned above could also include the signal sequence in the N-terminal:

(SEQ ID NO:11)

MGLSTVPDLLLPLVLLELLVGIYPSGVIGLVPHLGDREKR

Suitable peptides for trimerisation also include functional analogues of any of the peptides mentioned herein, including functional analogues of SEQ ID NOS:1-11.

The trimerisation molecules having a maleimide moiety are coupled to peptides via a Michael reaction with a free cysteine on the peptide. Thus, a suitable sequence could be a functional analogue wherein Cys has been added or exchanged with other amino acids in the sequences. Such analogues are specific aspects of the invention. The amino acids are preferentially added or exchanged in positions where they do not affect binding, but a suitable as handles for attaching the trimerisation molecule as demonstrated below.

For example, in an aspect of this invention, the peptide has the sequence of SEQ ID NO:2. The invention also provides nucleic acids encoding this sequence, including the following:

(SEQ ID NO:12)

gatagtgtgtgtccccaaggaaaatatatccaccctcaaaataattcgat
ttgctgtaccaagtgccacaaaggaacctacttgtacaatgactgtccag
gcccggggcaggatacggactgcagggagtgtgagagcggctccttcacc
gcttcagaaaaccacctcagacactgcctcagctgctccaaatgccgaaa
ggaaatgggtcaggtggagatctcttcttgcacagtggaccgggacaccg
tgtgtggctgcaggaagaaccagtaccggcattattggagtgaaaacctt
ttccagtgcttcaattgcagcctctgcctcaatgggaccgtgcacctctc
ctgccaggagaaacagaacaccgtgtgcaccaccagttgt

Any functional analogue of the above mentioned sequences, including mutated or fused analogues, can also be used for trimerisation.

In one aspect, the peptide is a TNFR fragment functioning as a substrate for the CPY reaction, forming an intermediate suitable for trimerisation. This is described in EP243929 and in WO2005035553. Such substrates can have a c-terminal alanine. The peptide having the sequence of SEQ ID NO:1 is an example of such a sequence. The invention also provides nucleic acids encoding this sequence, including the following:

(SEQ ID NO:13)

gatagtgtgtgtccccaaggaaaatatatccaccctcaaaataattcgat
ttgctgtaccaagtgccacaaaggaacctacttgtacaatgactgtccag
gcccggggcaggatacggactgcagggagtgtgagagcggctccttcacc
gcttcagaaaaccacctcagacactgcctcagctgctccaaatgccgaaa
ggaaatgggtcaggtggagatctcttcttgcacagtggaccgggacaccg
tgtgtggctgcaggaagaaccagtaccggcattattggagtgaaaacctt
ttccagtgcttcaattgcagcctctgcctcaatgggaccgtgcacctctc
ctgccaggagaaacagaacaccgtgtgcaccaccagtcac

Any functional analogues of the above mentioned sequences, including mutated or fused analogues, can also be used for trimerisation.

The activity of a monomer of TNFR can be represented by an ED50 of 10-200 nM. In an aspect of the invention, the trimerised molecules are at the same level of activity. In an aspect of the invention, the trimerised molecules are having an improved activity. In an aspect of the invention, the trimerisd molecules are having an ED50 of below 5 nM. In an aspect of the invention, the trimerised molecules are having an ED50 of below 1 nM. In an aspect of the invention, the trimerised molecule are having an ED50 below 0.1 nM. In an aspect of the above, the trimerised molecules are the sequences in any one of SEQ ID NOS:1-7.

TNFα is indicated in a number of other inflammatory diseases, e.g. psoriasis, sepsis, systemic lupus erythematosus, Hashimoto's thyroiditis, multiple sclerosis, myasthenia gravis, Guillain-Barré syndrome, autoimmune uveitis, Crohn's disease, ulcerative colitis, primary biliary cirrhosis, autoimmune hepatitis, autoimmune hemolytic anemia, pernicious anemia, autoimmune thrombocytopenia, Grave's disease, autoimmune oophoritis, autoimmune orchitis, temporal arteritis, anti-phospholipid syndrome, Wegener's granulomatosis, Behcet's disease, rheumatoid arthritis, scleroderma, polymyositis, dermatomyositis, ankylosing spondylitis, Sjogren's syndrome, dermatitis herpetiformis, pemphigus vulgaris, vitiligo, psoriatic arthritis, osteoarthritis, steroid-resistant asthma, chronic obstructive pulmonary disease, atherosclerosis, and transplant rejection. Thus, a trimerised molecule comprising a TNFα can be useful in the treatment of these diseases.

### Nucleic Acid Construct

As used herein the term “nucleic acid construct” is intended to indicate any nucleic acid molecule of cDNA, genomic DNA, synthetic DNA or RNA origin. The term “construct” is intended to indicate a nucleic acid segment which may be single- or double-stranded, and which may be based on a complete or partial naturally occurring nucleotide sequence encoding a polypeptide of interest. The construct may optionally contain other nucleic acid segments.

The nucleic acid construct of the invention encoding the polypeptide of the invention may suitably be of genomic or cDNA origin, for instance obtained by preparing a genomic or cDNA library and screening for DNA sequences coding for all or part of the polypeptide by hybridization using synthetic oligonucleotide probes in accordance with standard techniques.

The nucleic acid construct of the invention encoding the polypeptide may also be prepared synthetically by established standard methods, e.g. the phosphoramidite method described by Beaucage and Caruthers, Tetrahedron Letters 22 (1981), 1859-1869, or the method described by Matthes et al., EMBO Journal 3 (1984), 801-805. According to the phosphoramidite method, oligonucleotides are synthesized, e.g. in an automatic DNA synthesizer, purified, annealed, ligated and cloned in suitable vectors.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct may also be prepared by polymerase chain reaction using specific primers, for instance as described in U.S. Pat. No. 4,683,202 or Saiki et al., Science 239 (1988), 487-491.

In an exemplary embodiment, the nucleic acid construct of the invention comprises the DNA sequence of SEQ ID NOS:12 or 13, as well as other nucleic acid sequences encoding the amino acid sequences of SEQ ID NOS:1 or 2 but which differ from the DNA sequences of SEQ ID NOS:1 or 2 by virtue of the degeneracy of the genetic code.

The invention further encompasses nucleic acid sequences which hybridize to a nucleic acid molecule (either genomic, synthetic or cDNA or RNA) encoding the amino acid sequence of any one of SEQ ID NOS:1-7 under the conditions of high stringency as described. The homologue of the polypeptide may be one encoded by a nucleotide sequence hybridizing with an oligonucleotide probe prepared on the basis of the nucleotide sequence of SEQ ID NO:12 or 13 or on another nucleic acid encoding the amino acid sequence of any one of SEQ ID NOS:1-7. Such conditions include, for example, hybridization under medium to high stringency, presoaking in 5×SSC and prehydbridizing for 1 hr. at about 40° C. in a solution of 20% formamide, 5×Denhardt's solution, 50 mM sodium phosphate, pH 6.8, and 50 μg denatured sonicated calf thymus DNA, followed by hybridization in the same solution supplemented with the labelled oligonucleotide probe for 18 hrs. at about 40° C., followed by a wash in 0.4×SSC at a temperature of about 45° C.

Molecules to which the oligonucleotide probe hybridizes under these conditions can be detected using standard detection procedures (e.g. Southern blotting).

The nucleic acid construct is preferably a DNA construct which term will be used exclusively in the following.

### Recombinant Vector

In a further aspect, the present invention relates to a recombinant vector comprising a DNA construct of the invention. The recombinant vector into which the DNA construct of the invention is inserted may be any vector which may conveniently be subjected to recombinant DNA procedures, and the choice of vector will often depend on the host cell into which it is to be introduced. Thus, the vector may be an autonomously replicating vector, i.e. a vector which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the polypeptide of the invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

The promoter may be any DNA sequence which shows transcriptional activity in the host cell of choice and may be derived from genes encoding peptides either homologous or heterologous to the host cell.

Examples of suitable promoters for directing the transcription of the DNA encoding the polypeptide of the invention in mammalian cells are the SV40 promoter (Subramani et al., Mol. Cell. Biol. 1 (1981), 854-864), the MT-1 (metallothionein gene) promoter (Palmiter et al., Science 222 (1983), 809-814) or the adenovirus 2 major late promoter.

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. No. 5,155,037; U.S. Pat. No. 5,162,222), or the baculovirus 39K delayed-early gene promoter (U.S. Pat. No. 5,155,037; U.S. Pat. No. 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al., eds.), Plenum Press, New York, 1982), or the TPI1 (U.S. Pat. No. 4,599,311) or ADH2-4-c (Russell et al., Nature 304 (1983), 652-654) promoters.

Examples of suitable promoters for use in filamentous fungus host cells are, for instance, the ADH3 promoter (McKnight et al., EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral α-amylase, A. niger acid stable α-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters.

Examples of suitable promoters for use in bacterial host cells include the promoter of the Bacillus stearothermophilus maltogenic amylase gene, the Bacillus licheniformis alphaamylase gene, the Bacillus amyloliquefaciens BAN amylase gene, the Bacillus subtilis alkaline protease gen, or the Bacillus pumilus xylosidase gene, or by the phage Lambda P_{R} or P_{L} promoters or the E. coli lac, trp or tac promoters.

The DNA sequence encoding the polypeptide of the invention may also, if necessary, be operably connected to a suitable terminator, such as the human growth hormone terminator (Palmiter et al., op. cit.) or (for fungal hosts) the TPI1 (Alber and Kawasaki, op. cit.) or ADH3 (McKnight et al., op. cit.) terminators. The vector may further comprise elements such as polyadenylation signals (e.g. from SV40 or the adenovirus 5 Elb region), transcriptional enhancer sequences (e.g. the SV40 enhancer) and translational enhancer sequences (e.g. the ones encoding adenovirus VA RNAs).

The recombinant vector of the invention may further comprise a DNA sequence enabling the vector to replicate in the host cell in question. An example of such a sequence (when the host cell is a mammalian cell) is the SV40 origin of replication.

When the host cell is a yeast cell, suitable sequences enabling the vector to replicate are the yeast plasmid 2μ replication genes REP 1-3 and origin of replication.

The vector may also comprise a selectable marker, e.g. a gene the product of which complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For filamentous fungi, selectable markers include amdS, pyrG, argB, niaD, sC.

To direct a polypeptide of the present invention into the secretory pathway of the host cells, a secretory signal sequence (also known as a leader sequence, prepro sequence or pre sequence) may be provided in the recombinant vector. The secretory signal sequence is joined to the DNA sequence encoding the polypeptide in the correct reading frame. Secretory signal sequences are commonly positioned 5′ to the DNA sequence encoding the polypeptide. The secretory signal sequence may be that normally associated with the polypeptide or may be from a gene encoding another secreted peptide.

For secretion from yeast cells, the secretory signal sequence may encode any signal peptide which ensures efficient direction of the expressed polypeptide into the secretory pathway of the cell. The signal peptide may be naturally occurring signal peptide, or a functional part thereof, or it may be a synthetic peptide. Suitable signal peptides have been found to be the α-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the polypeptide. The function of the leader peptide is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The leader peptide may be the yeast α-factor leader (the use of which is described in e.g. U.S. Pat. No. 4,546,082, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease, a Humicola lanuginosa lipase. The signal peptide is preferably derived from a gene encoding A. oryzae TAKA amylase, A. niger neutral α-amylase, A. niger acid-stable amylase, or A. niger glucoamylase.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the present polypeptide, the promoter and optionally the terminator and/or secretory signal sequence, respectively, and to insert them into suitable vectors containing the information necessary for replication, are well known to persons skilled in the art (cf., for instance, Sambrook et al., op.cit.).

### Host Cells

The DNA sequence encoding the present polypeptide introduced into the host cell may be either homologous or heterologous to the host in question. If homologous to the host cell, i.e. produced by the host cell in nature, it will typically be operably connected to another promoter sequence or, if applicable, another secretory signal sequence and/or terminator sequence than in its natural environment. The term “homologous” is intended to include a cDNA sequence encoding a polypeptide native to the host organism in question. The term “heterologous” is intended to include a DNA sequence not expressed by the host cell in nature. Thus, the DNA sequence may be from another organism, or it may be a synthetic sequence.

The host cell into which the DNA construct or the recombinant vector of the invention is introduced may be any cell which is capable of producing the present polypeptide and includes bacteria, yeast, fungi and higher eukaryotic cells.

Examples of bacterial host cells which, on cultivation, are capable of producing the polypeptide of the invention are grampositive bacteria such as strains of Bacillus, such as strains of B. subtilis, B. licheniformis, B. lentus, B. brevis, B. stearothermophilus, B. alkalophilus, B. amyloliquefaciens, B. coagulans, B. circulans, B. lautus, B. megatherium or B. thuringiensis, or strains of Streptomyces, such as S. lividans or S. murinus, or gramnegative bacteria such as Echerichia coli. The transformation of the bacteria may be effected by protoplast transformation or by using competent cells in a manner known per se (cf. Sambrook et al., supra).

When expressing the polypeptide in bacteria such as E. coli, the polypeptide may be retained in the cytoplasm, typically as insoluble granules (known as inclusion bodies), or may be directed to the periplasmic space by a bacterial secretion sequence. In the former case, the cells are lysed and the granules are recovered and denatured after which the polypeptide is refolded by diluting the denaturing agent. In the latter case, the polypeptide may be recovered from the periplasmic space by disrupting the cells, e.g. by sonication or osmotic shock, to release the contents of the periplasmic space and recovering the polypeptide.

Examples of suitable mammalian cell lines are the COS (ATCC CRL 1650), BHK (ATCC CRL 1632, ATCC CCL 10), CHL (ATCC CCL39) or CHO (ATCC CCL 61) cell lines. Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous polypeptides therefrom are described, e.g. in U.S. Pat. No. 4,599,311, U.S. Pat. No. 4,931,373, U.S. Pat. Nos. 4,870,008, 5,037,743, and U.S. Pat. No. 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequence encoding the polypeptide of the invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of peptides is described in, e.g., EP 272 277 and EP 230 023. The transformation of F. oxysporum may, for instance, be carried out as described by Malardier et al. (Gene 1989; 78:147-156).

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. No. 4,745,051; U.S. Pat. No. 4,879,236; U.S. Pat. Nos. 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting the expression of the present polypeptide, after which the resulting polypeptide is recovered from the culture.

The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection). The polypeptide produced by the cells may then be recovered from the culture medium by conventional procedures including separating the host cells from the medium by centrifugation or filtration, precipitating the proteinaceous components of the supernatant or filtrate by means of a salt, e.g. ammonium sulphate, purification by a variety of chromatographic procedures, e.g. ion exchange chromatography, gelfiltration chromatography, affinity chromatography, or the like, dependent on the type of polypeptide in question.

### Modified Peptides or Peptide Trimers

Unless otherwise stated or clearly contradicted by context, the terms peptide and peptided trimer also generally encompasses derivatised peptide or peptide trimer molecules (“derivatives”). In a “derivative”, one or more of the amino acid residues of a peptide have been chemically modified (e.g., by alkylation, acylation, ester formation, or amide formation) or associated with one or more non-amino acid organic and/or inorganic atomic or molecular substituents such as, e.g., a polyethylene glycol (PEG) group, a lipophilic substituent, a fluorophore, biotin, a radionuclide, or other atom or molecule. These substituents may optionally be linked to the amino acid sequence of the peptide by a spacer residue or group such as β-alanine, gamma-aminobutyric acid (GABA), L/D-glutamic acid, succinic acid, and the like). Also or alternatively, the peptide derivative can comprise non-essential, non-naturally occurring, and/or non-L amino acid residues. Non-limiting examples of unusual amino acid residues include, for example, 2-aminoadipic acid; 3-Aminoadipic acid; β-Alanine; β-aminopropionic acid; 2-Aminobutyric acid; 4-Aminobutyric acid; 6-Aminocaproic acid; 2-Aminoheptanoic acid; 2-Aminoisobutyric acid; 3-Aminoisobutyric acid, 2-Aminopimelic acid; 2,4-Diaminobutyric acid; Desmosine; 2,2′-Diaminopimelic acid; 2,3-Diaminopropionic acid; N-Ethylglycine; N-Ethylasparagine; Hydroxylysine; allo-Hydroxylysine; 3-Hydroxyproline; 4-Hydroxyproline; Isodesmosine; allo-Isoleucine; N-Methylglycine; N-Methylisoleucine; 6-N-Methyllysine; N-Methylvaline; Norvaline; Norleucine; Ornithine; and others.

### Pharmaceutical Compositions

Another object of the present invention is to provide a pharmaceutical formulation comprising a compound of the invention or optionally together with any other compound mentioned in the present application which is present in a concentration from 0.1 mg/ml to 100 mg/ml, and wherein said formulation has a pH from 2.0 to 10.0. The formulation may further comprise a buffer system, preservative(s), tonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a compound of the invention, or any other compound as mentioned above and a buffer, wherein said compound is present in a concentration from 0.1 mg/ml or as mentioned above, preferably from 0.5 mg/ml-50 mg/ml and wherein said formulation has a pH from about 2.0 to about 10.0. Preferred pH is from 3.0 to about 8.0. Particular preferred range is from 4.0-6.0, such as for example the ranges 4.0-4.5, 4.5-5.0, 5.0-5.5 and 5.5-6.0.

In another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable antimicrobial preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomersal, bronopol, benzoic acid, imidurea, chlorohexidine, sodium dehydroacetate, chlorocresol, ethyl p-hydroxybenzoate, benzethonium chloride, chlorphenesine (3p-chlorophenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabilizer. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form or a mixture thereof. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L or D isomer) of a particular amino acid (e.g., methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the peptide.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L or D isomer) or combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabilizer selected from the group of high molecular weight polymers or low molecular compounds. In a further embodiment of the invention the stabilizer is selected from polyethylene glycol (e.g., PEG 3350), polyvinyl alcohol (PVA), polyvinylpyrrolidone, carboxy/hydroxycellulose or derivates thereof (e.g., HPC, HPC-SL, HPC-L and HPMC), cyclodextrins, sulphur-containing substances as monothioglycerol, thioglycolic acid and 2-methylthioethanol, and different salts (e.g., sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g., Tween-20, Tween-40, Tween-80 and Brij-35), monoglycerides or ethoxylated derivatives thereof, diglycerides or polyoxyethylene derivatives thereof, alcohols, glycerol, lectins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g., lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g., sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g., N-alkyl-N,N-dimethylammonio-1-propanesulfonates, 3-cholamido-1-propyldimethylammonio-1-propanesulfonate, cationic surfactants (quaternary ammonium bases) (e.g., cetyl-trimethylammonium bromide, cetylpyridinium chloride), non-ionic surfactants (eg. Dodecyl β-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

Other ingredients may also be present in the peptide pharmaceutical formulation of the present invention. Such additional ingredients include wetting agents, emulsifiers, antioxidants, bulking agents, tonicity modifiers, chelating agents, metal ions, oleaginous vehicles, peptides (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound of the invention or any other compound as mentioned above according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of a compound of the invention or any other compound as mentioned above, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of a compound of the invention or any other compound as mentioned above using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of a compound of the invention or any other compound as mentioned above, in the form of a nasal or pulmonal spray. As a still further option, the pharmaceutical compositions containing a compound of the invention or any other compound as mentioned above can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the peptide formulation as used herein refers to the tendency of the peptide to form biologically inactive and/or insoluble aggregates of the peptide as a result of exposure of the peptide to thermo-mechanical stresses and/or interaction with interfaces and surfaces that are destabilizing, such as hydrophobic surfaces and interfaces. Physical stability of the aqueous peptide formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physically unstable with respect to peptide aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradation pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

### General Methods of Preparation

### General Procedure (A)

### Step A:

The molecules required for the trimerisation of peptides and proteins are built up from amines and acids via the formation of an amide bond. In the case there is involved a di- or trifunctional molecule in which only one group is to react, the other groups can be protected using standard protecting groups.

The synthesis starts with the mono protection of the diamine building block, which is represented by A in the general formula, using procedures known to those skilled in the art (e.g. T. W. Greene, P. G. M. Wuts, Protective groups in organic synthesis, 2^{nd} ed., 1991 John Wiley & Sons, Inc. New York).

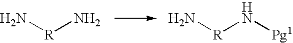

The diacid building block, represented by B in the general formula, can be protected using procedures known to those skilled in the art (e.g. T. W. Greene, P. G. M. Wuts, Protective groups in organic synthesis, 2^{nd} ed., 1991 John Wiley & Sons, Inc. New York).

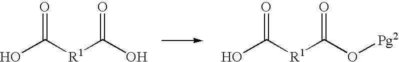

### Step B:

The next step involves the amide bond formation between the different building blocks. For example, the central trifunctional acid is coupled with three mono protected diamines using standard amide bond formation conditions known to those skilled in the art. Preferable the acid is activated using a carbodiimide such as, e.g., diisopropylcarbodiimide and an active ester is formed such as, e.g., a benzotriazolyl ester. The active ester is then reacted with the monoprotected diamine:

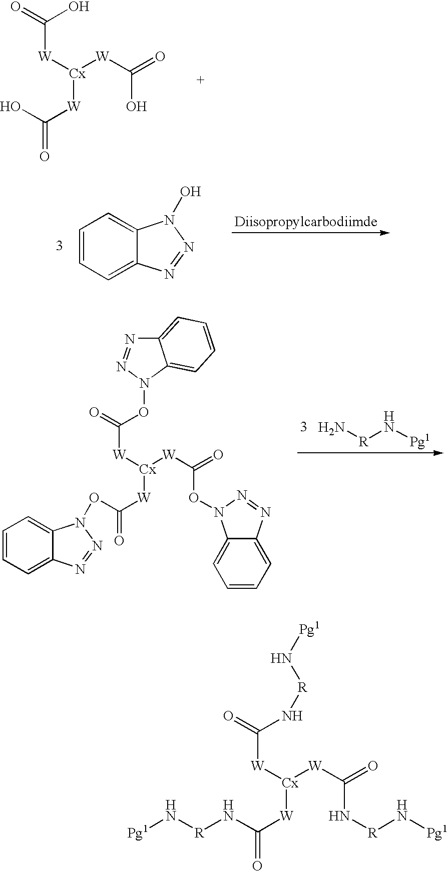

The reaction between an active ester and an amine can be promoted by the addition of a tertiary amine, e.g. diisoproylethylamin or triethylamine.

The protecting groups are removed with standard methods known to those skilled in the art (e.g. T. W. Greene, P. G. M. Wuts, Protective groups in organic synthesis, 2^{nd} ed., 1991 John Wiley & Sons, Inc. New York) before the triamine is allowed to react further with a mono protected mono activated diacid, B, or an activated acid containing a moiety (e.g. β-maleimido propionic acid) which terminates the arm and allows the molecule to react with a functional group on the peptide or protein, referred to as Y in the general formula. The reaction below demonstrates the incorporation of a C moiety:

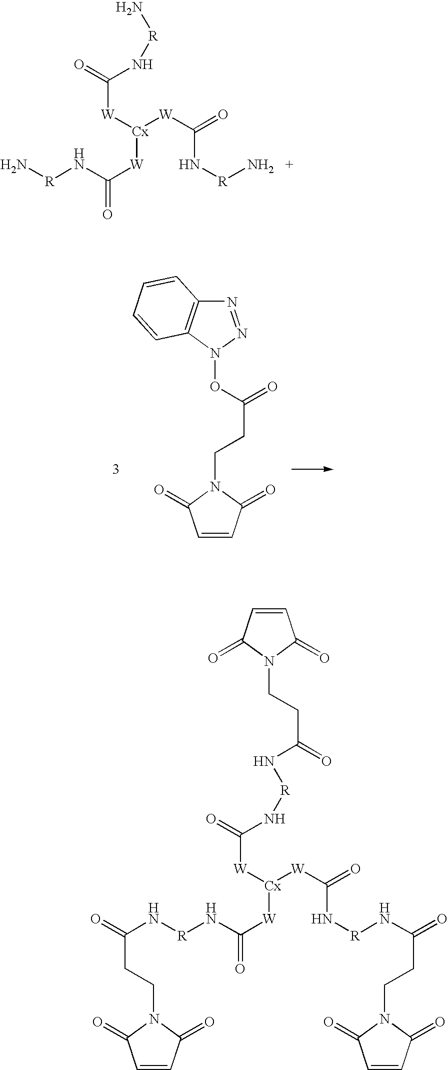

The activation and reaction of the acid is known to those skilled in the art and has be described earlier in this procedure. Prior to setting the terminating group on the arms, the arms can be elongated by subsequent attachments of diacids, B, and diamines, A, using the procedure here described.

General Procedure (B)

A sulfanyl-pyrrolidine-2,5-dione moiety may be formed by dissolving the peptide containing a thiol in question in water. Organic solvents may be added to increase solubility.

The solution is buffered to a suitable pH-value such as e.g. between pH 0 and pH 10, between pH 3 and pH 6, or pH 5 and kept at a suitable temperature such as e.g. 0-60° C. The maleimide in question is added, and the sulfanyl-pyrrolidine-2,5-dione moiety is formed according to the reaction scheme below:

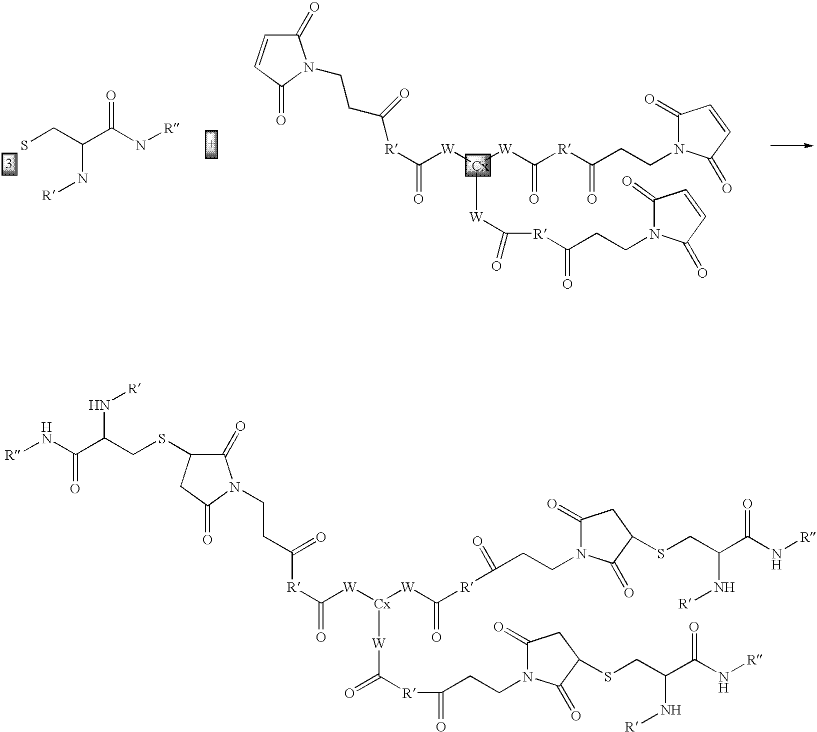

The pH of choice is determined, e.g., by the solubility of the peptide to be used. The solubility of peptides is to a large extent determined by the pKa of the peptide. Normally, the solubility of a given peptide is at its minimum when pH equals pKa of the peptide. At pH values between 7-14 other nucleophilic amino acid sidechains can react with the maleimide moiety and it lies within the skills of a skilled person to select a pH at which to run the reaction taking due care to the above consideration.

### General Procedure (C)

An oxime moiety may be formed by dissolving the transacylated peptide in question as described in Danish Patent application PA 2003 01496, now published as WO2005035553, which contains an unnatural amino acid, N-terminal acid or C-terminal amine containing a keto or aldehyde functionality, in water. Organic solvents may be added to increase solubility. The solution is buffered to a suitable pH-value such as e.g. between pH 0 and pH 10, between pH 3 and pH 6, or pH 5 and kept at a suitable temperature such as e.g. 0-60° C. The hydroxylamine in question is added, and the oxime moiety is formed according to the reaction scheme below:

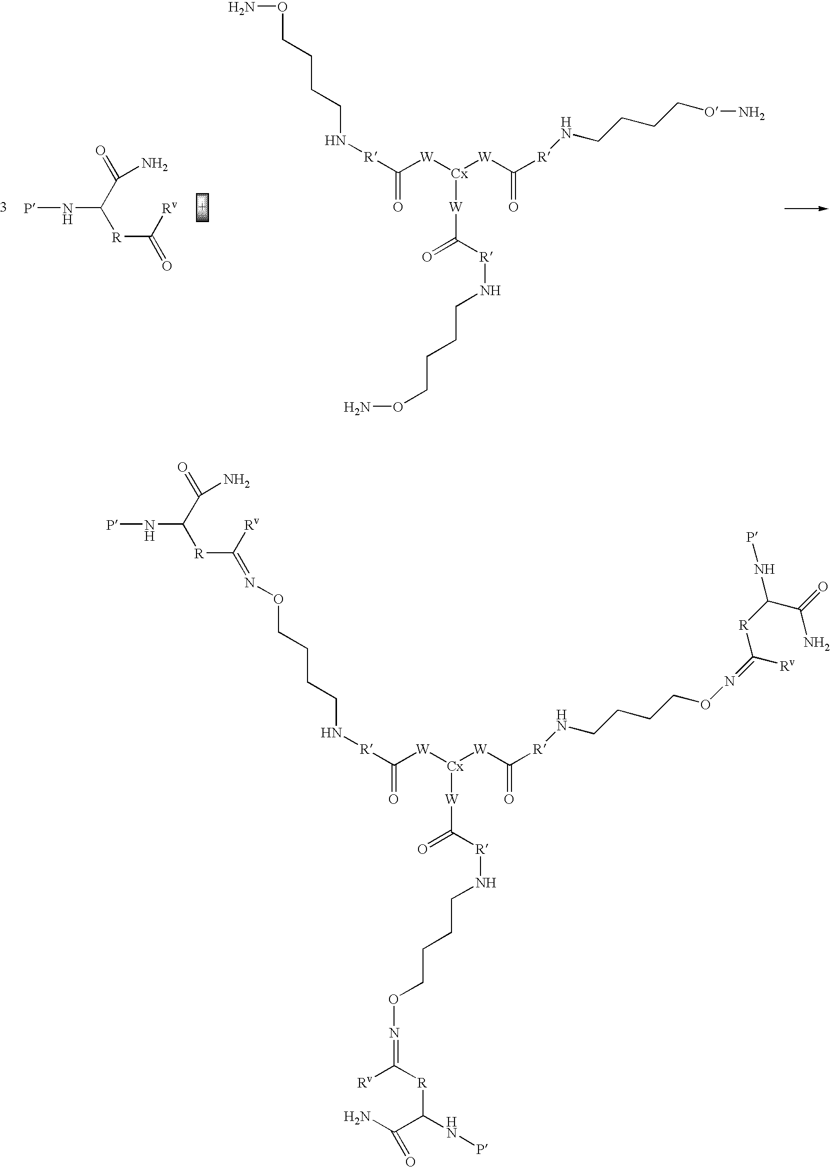

The pH of choice is determined, e.g., by the solubility of the peptide to be used. The solubility of peptides is to a large extent determined by the pKa of the peptide. Normally, the solubility of a given peptide is at its minimum when pH equals pKa of the peptide. It lies within the skills of a skilled person to select a pH at which to run the reaction taking due care to the above considerations.

### EXAMPLE 1

### Synthesis of a Trimerisation Molecule

### Step 1. Monoprotection of a Diamine.

50 g of the diamine (4,7,10-Trioxa1,13-Tridecanediamine) was dissolved in 250 ml of dichlormethane. 17.5 mL of trifluoroacetic acid was added dropwise to the clear colorless solution. 49.5 g of di-tertbutyl-dicarbonate in 2000 ml of dichlormethane was added dropwise over 5 hours to the solution. The reaction mixture was stirred over night at room temperature and then concentrated in vacuum, yielding of clear yellow oil.

The oil was dissolved in dichlormethane and the desired compound was extracted into 2×0.1N HCl (250 ml).

The water phase was treated with 280 ml 1N NaOH. The basic water phase was extracted twice with 250 ml of dichlormethane. The combined organic phases were washed with brine (200 ml). The dichloromethane was evaporated in vacuum yielding a clear light yellow oil.

### Step 2. Attachment of the Mono-Boc-Amine to the Central Unit.

1 g of nitrilotriacetic acid was dissolved in 15 mL of tetrahydrofuran. 4 g of 1-hydroxybenzotriazole (HOBt) and 2.6 mL of diisopropylcarbodiimide (DIC) were added and the mixture was stirred for 30 min. 5.4 g of the mono-boc-amine and 2.7 mL of diisopropylethylamine (DIPEA) were added to the active ester. The reaction mixture was stirred over night at room temperature.

The reaction mixture was concentrated in vacuum and apportioned between water and ethyl acetate. The ethyl acetate phase was concentrated in vacuo yielding a semicrystalline oil in 10 g. The oil was chromatographed on Silica with 30% methanol in dichlormethane. The oil passed through the column as a plug. The fraction was concentrated in vacuum, and the remanence was redissolved in ethyl acetate. The solution was washed with sat. sodium hydrogen carbonate and finally with sat. sodium hydrogen sulphate. The ethyl acetate phase was concentrated in vacuo and then stripped with acetonitrile.

The remaining oil was dissolved in 100 mL of 50% trifluoroacetic acid in dichlormethane and then stirred at room temperature for 1 hour. The reaction mixture was concentrated in vacuo.

The mixture was apportioned between water and ethyl acetate. The water phase was washed twice with ethyl acetate and then lyophilised yielding a yellow oil in 9.5 g.

### Step 3. Attachment of the Maleimide Moiety.

74 mg of β-maleimidopropionic acid was dissolved in 6 mL of dichloromethane and 0.18 mL of triethylamine was added. The clear colorless solution was cooled to approx. 0° C. on ice/water. 0.11 mL of pivaoylchloride was dissolved in 3 mL dichlormethane and then added dropwise to the cold solution.

The reaction mixture was stirred after addition without cooling for 1 hour. The red solution was concentrated to dryness and redissolved in dichlormethane.

This clear red solution was added to a solution of 100 mg of the amine from step 4 and 0.18 mL of triethylamine in dichloromethane.

The clear red reaction mixture was stirred over night at room temperature.

The solvent was removed in vacuum and the residual oil was purified using a RP-HPLC.

### EXAMPLE 2

### Trimerisation of Albumin

The compound prepared according to example 1, was reacted with albumin according to the described procedure:

33 mg Albagen (recombinant human albumin) was dissolved in 660 μL (˜1 mmM) and portionated into five times 100 μL.

Added to Sample
1: nothing.
2: 10 μL 30 mM trimerisation molecule of example 1.
3: 10 μL 15 mM trimerisation molecule of example 1.
4: 10 μL 7.5 mM trimerisation molecule of example 1.
5: 10 μL 3.75 mM trimerisation molecule of example 1.

The samples were vortexed and then kept at room temperature for one hour. An SDS-gel shows the formation of a trimer after 1 hour.

### EXAMPLE 3

### Trimerisation of MC4 ligand Nitrilotris-{4-[4-(2-{[3-(2-{2-[3-(acetylamino)-propoxy]-ethoxy}-ethoxy)propylcarbamoyl]-methoxy}-acetylamino)-butoxyimino]-cyclohexanecarbonyl-Gly-Ser-Gln-His-Ser-Nle-c[Glu-Hyp-D-Phe-Arg-Trp-Lys]-NH₂}(SEQ ID NO:14)

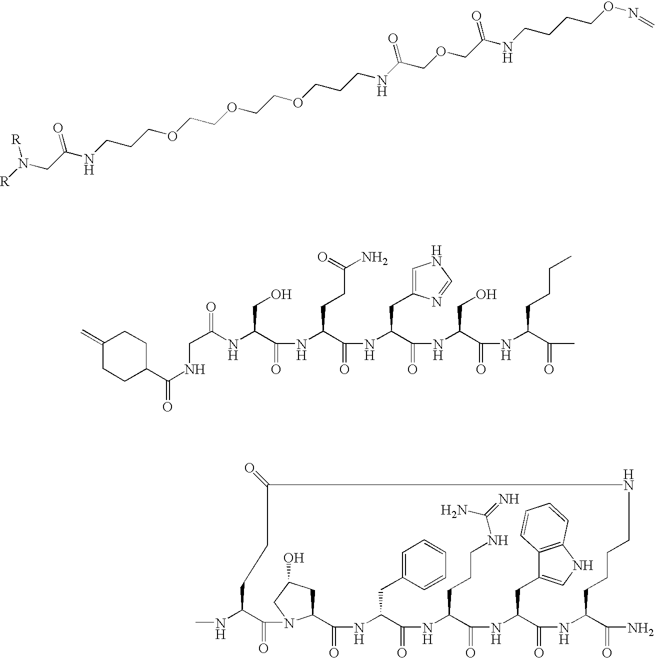

In the drawing the two R represent the other two arms of the trimer, which have been collapsed in order to be able to show one arm in a reasonable scale.

### Step 1: Synthesis of the Peptide

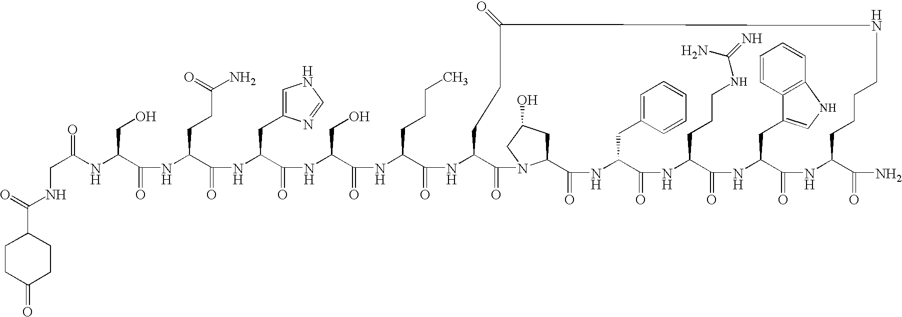

1.a. The protected peptidyl resin 4-oxocyclohexanecarbonyl-Gly-Ser(tBu)-Gln(Trt)-His(Trt)-Ser(tBu)-Nle-Glu(2-phenylisopropyloxy)-Hyp(tBu)-D-Phe-Arg(Pmc)Trp(Boc)-Lys(Mtt)-(Rink resin) was synthesized according to the Fmoc strategy on an Applied Biosystems 431 A peptide synthesizer on a 0.25 mmol scale using the manufacturer-supplied “FastMoc UV” protocols which employ HBTU (2-(1H-Benzotriazol-1-yl-)-1,1,3,3 tetramethyluronium hexafluorophosphate) mediated couplings in NMP (N-methylpyrrolidone) and UV monitoring of the deprotection of the Fmoc protection group. The starting resin used for the synthesis was 0.50 g (4-((2′,4′-dimethoxyphenyl)-(Fmoc-amino)methyl)-phenoxy-polystyrene resin (Rink resin) (Novabiochem) with a loading of 0.51 mmol/g. The protected amino acid derivatives used were Fmoc-Lys(Mtt)-OH, Fmoc-Trp(Boc)-OH, Fmoc-Arg(Pmc)OH, Fmoc-D-Phe-OH, Fmoc-Hyp(tBu)-OH, Fmoc-Glu(2-phenylisopropyloxy)-OH and Fmoc-Nle-OH.

1.b The resin resulting from (1.a) was treated with 5×10 ml 2% trifluoroacetic acid (TFA), 2% triethylsilane (TES) in DCM during 60 minutes with regular mixing. The resin was washed with NMP, NMP with 5% DIEA and NMP. The peptide was cyclized using HOBt (1.0 mmol), (benzotriazol-1-yloxy)tripyrrolidinophosphonium hexafluorophosphate 1H-benzotriazol-1-yloxytripyrrolidinophosphonium hexafluorophosphate (PyBOP) (1.0 mmol) and DIEA (2.0 mmol) in NMP (5 ml) with regular mixing for 4 h. The resin was washed with NMP and DCM.

1.c The peptide was cleaved from the resin obtained from (1.c) by stirring for 60 minutes at room temperature with 10 ml of 2.5% water and 2.5% TES in TFA. The cleavage mixture was filtered and the filtrate was concentrated to approximately 1 ml by a stream of nitrogen. The crude peptide was precipitated from this oil with 50 ml diethyl ether and washed 3 times with 50 ml diethyl ether.

The crude cyclic peptide was purified by preparative RP-HPLC.

### Step 2: Synthesis of the Trimerisation Moiety

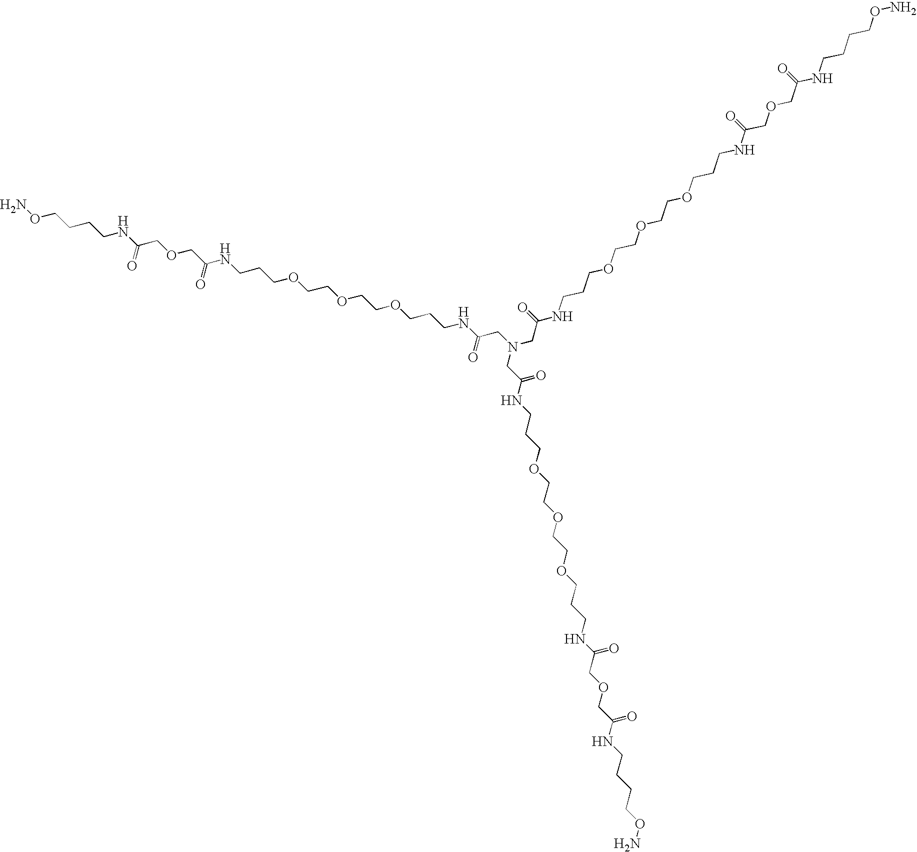

### 2.a Monoprotection of a Diamine.

50 g of the diamine (4,7,10-Trioxa1,13-Tridecanediamine) was dissolved in 250 ml of dichlormethane. 17.5 mL of trifluoroacetic acid was added dropwise to the clear colorless solution. 49.5 g of di-tertbutyl-dicarbonate in 2000 ml of dichlormethane was added dropwise over 5 hours to the solution. The reaction mixture was stirred over night at room temperature and then concentrated in vacuum, yielding of clear yellow oil.

The oil was dissolved in dichlormethane and the desired compound was extracted into 2×0.1N HCl (250 ml).

The water phase was treated with 280 ml 1N NaOH. The basic water phase was extracted twice with 250 ml of dichlormethane. The combined organic phases were washed with brine (200 ml). The dichloromethane was evaporated in vacuum yielding a clear light yellow oil.

### 2.b Attachment of the Mono-Boc-Amine to the Central Unit.

1 g of nitrilotriacetic acid was dissolved in 15 mL of tetrahydrofuran. 4 g of 1-hydroxybenzotriazole (HOBt) and 2.6 mL of diisopropylcarbodiimide (DIC) were added and the mixture was stirred for 30 min. 5.4 g of the mono-boc-amine and 2.7 mL of diisopropylethylamine (DIPEA) were added to the active ester. The reaction mixture was stirred over night at room temperature.

The reaction mixture was concentrated in vacuum and apportioned between water and ethyl acetate. The ethyl acetate phase was concentrated in vacuo yielding a semicrystalline oil in 10 g. The oil was chromatographed on Silica with 30% methanol in dichlormethane. The oil passed through the column as a plug. The fraction was concentrated in vacuum, and the remanence was redissolved in ethyl acetate. The solution was washed with sat. sodium hydrogen carbonate and finally with saturated sodium hydrogen sulphate. The ethyl acetate phase was concentrated in vacuo and then stripped with acetonitrile.

The remaining oil was dissolved in 100 mL of 50% trifluoroacetic acid in dichlormethane and then stirred at room temperature for 1 hour. The reaction mixture was concentrated in vacuo.

The mixture was apportioned between water and ethyl acetate. The water phase was washed twice with ethyl acetate and then lyophilised yielding a yellow oil in 9.5 g.

### 2.c Creation of the Anchoring Unit.

6 g of (4-aminobutoxy)carbamic acid 1,1-dimethylethyl ester (WO 2005014049 A2) was dissolved in 20 mL pyridine and 4.1 g diglycolic anhydride was added. The clear yellow solution was stirred at 100° C. for 3 hours. The reaction mixture was then poured into 0.5 N HCl (150 mL) and the water phase was extracted with dichloromethane. The organic phase was dried over magnesium sulfate and the solvent was removed in vacuum yielding a yellow oil.

### 2.d Attachment of the Anchoring Unit to the Trimerisation Moiety

0.66 g of the acid from 2.c was dissolved in 5 mL dichloromethane and 0.29 mL triethylamine was added. The solution was cooled on ice and 0.25 mL pivaloyl chloride in 5 mL dichlormethane was added. The mixture was stirred for 1 hour. 0.53 g of the tetramine from 2.b was dissolved in 5 mL together with 0.29 mL triethylamine. To this solution the mixed anhydride was added and the mixture was stirred over night.

The solvent was removed in vacuum and the yielding oil was purified by HPLC on reverse phase using acetonitril/water+0.1% TFA. The boc-protecting groups were removed by treatment with 10% TFA in dichlormethane. The final product can be purified by reverse phase HPLC. Preferable the molecule is deprotected just before the trimerisation and used unpurified.

### Step 3: Trimerisation of the Peptide

3.a 21 mg of the peptide (1.c) was dissolved in 2 mL water and this solution was added to 6 mg of the unprotected trisoxiamine (2.d). The mixture was stirred for 1 hour at room temperature. The final product was then purified on reverse phase HPLC with acetonitril/water/0.1% TFA. MS-data: (M+4H)⁴⁺=1533.

### EXAMPLE 4

### Trimerisation of hGH

This example describes the trimerisation of Ser-hGH (SEQ ID NO:15).

The following buffers were used: Buffer A: 135 μL triethanolamine+290 μL 3-methylthiopropanol in 20 mL water. Buffer B: 48.1 mg NalO4 in 1 mL. Buffer C, 1.2 mL 3-methylthiopropanol in 80 mL water.

12 mg of Ser-hGH was dissolved in 1.2 mL buffer A. 125 μL buffer B was added the sample was vortexed. After 30 min the protein was filtered using a 10000 MW cutoff filter and washed 5 times with 10 mL buffer C.

The Boc-protecting group was cleaved from 1 mg Boc-Tristar (See example 3, step 2) with 1 mL TFA. The TFA was removed in a nitrogen stream after 30 min, and the Tristar taken up in 1 mL water and 10 μL TFA.

100 μL of the Tristar solution was added to 600 μL of the oxidised Ser-hGH solution. The pH was adjusted with DIPEA (diisopropylethylamine) to about 4-5. Trimerisation occurred within minutes.

A sample was analysed using a Bioanalyzer 2100 from Agilent. The SDS-Page revealed the formation of hGH dimer and trimer.

### EXAMPLE 5

### Trimerisation of Insulin

This example describes the trimerisation of human insulin (sequence of A chain: SEQ ID NO:16; sequence of B-chain: SEQ ID NO:17). The B-chain was derivatised with activated 3-Benzoylpropionic acid to B1-(3-benzoylpropionyl)-insulin.

Step 1: Synthesis of activated 3-Benzoylpropionic Acid

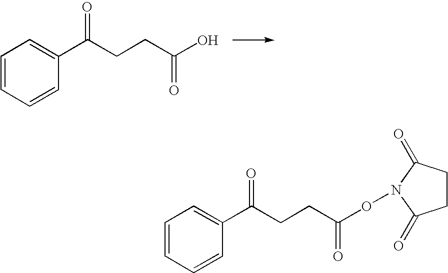

1 g of 3-Benzoylpropionic acid was dissolved in 20 ml of tetrahydrofuran (THF). 1.15 ml of diisopropylethyl-amine (DIPEA) was added and the mixture was cooled to 0° C. 2.0 g of 2-Succinimido-1,1,3,3-tetra-methyluronium tetrafluoroborate (TSTU) was added and the mixture was stirred for 30 min at 0° C.

The stirring was continued at ambient temperature over night. A precipitate was formed upon stirring. The precipitate was collected on a filter. The filtrate was concentrated in vacuo and redissolved in ethyl acetate. The organic phase was washed with 0.5 N HCl (3×25 ml) and with saturated sodium hydrogen carbonate (3××25 ml). The organic phase was concentrated in vacuo. The powder was suspended in ethyl acetate and the solid was collected on a filter funnel. The solid was dried in vacuo at ambient temperature.

### Step 2: Derivatisation of Insulin

100 mg of A1,B29-diBoc humaninsulin was dissolved in 2 mL of DMSO (dimethylsulfoxide) and 28 μl of TEA (triethylamine) was added. 5.5 mg of the activated 3-Benzoylpropionic acid dissolved in THF (1 ml) was added and the reaction mixture was stirred for 4.75 hours at ambient temperature.

The reaction mixture was cooled on ice bath and 5 ml of water was added. The pH was adjusted to pH=5.2 with 1 N HCl. The peptide was precipitated at 5° C. for 1 hour, isolated by centrifugation, and stored in a freezer over night.

The peptide was then treated with TFA (10 ml) for 15 minutes, and poured into ice-cooled diethyl ether (35 ml). The precipitate was isolated by centrifugation.

### Step 3: Trimerisation

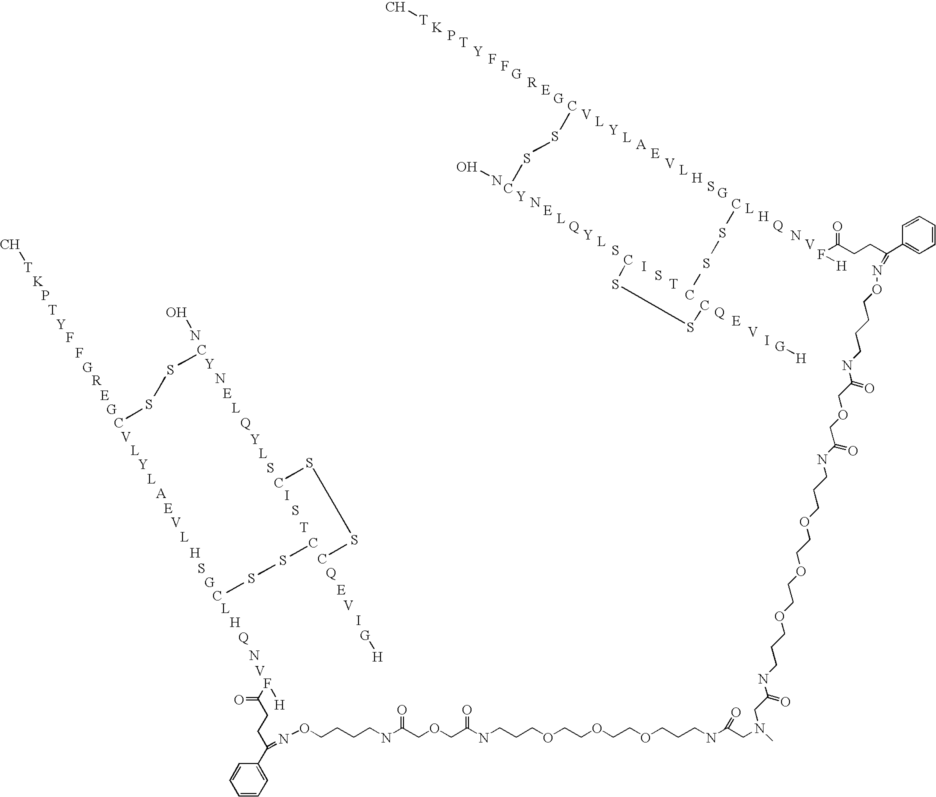

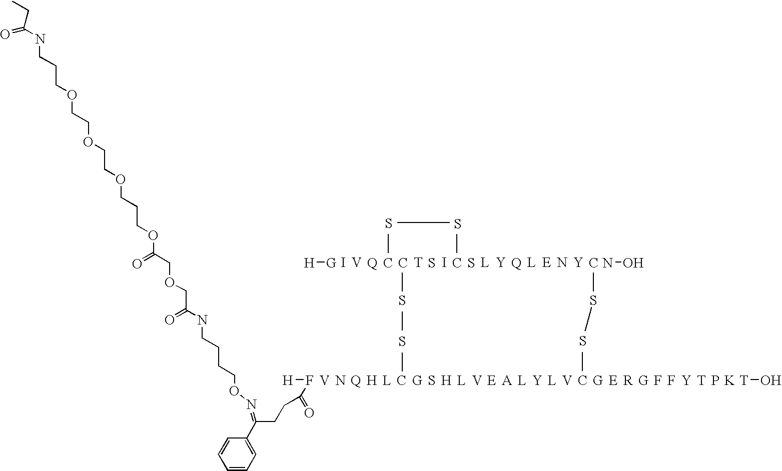

8.4 mg of the boc-protected trimerisation molecule was dissolved in DCM containing 10% of trifluoroacetic acid (TFA) and was stirred for 15 min. The solvent was removed by nitrogen air flow.

The remanence was dissolved in 1000 μL DMSO, and 840 μL of this solution was transferred to the insulin derivate from above. pH was adjusted to approx 5 with 1 N NaOH. The reaction mixture was stirred at ambient temperature for 60 hours. 500 μL of deionized water was added to the reaction mixture and stirring was continued for 96 hours.

An SDS-page gel revealed the formation of insulin dimer and trimer.

### Pharmacological Methods

Measurements of Affinity and Efficacy of Trimeric TNFR Fragments

Affinity can be measured as non limiting examples by competing with TNF in a TNF induced cytotoxicity assay in L292 or WEHI 164 cells or in a scintillation proximity assay based on coupling a known TNF a binding protein and competing for binding of 125I-labelled TNFα.

### Assay (II)

The effect in of the compounds of the present invention can be tested in the following assays:

Collagen-Induced RA.

The model has been described in the following papers:

Torbecke G J et al., Involvement of endogenous tumour necrosis factor a and trans-forming growth factor beta during inductiom of collagen type II arthritis in mice. 1992 PNAS, 89:7375-79

Williams R O et al., Anit-tumor necrosis factor ameliorates joint disease in murine collagen induced arthritis. 1992 PNAS, 89:9784-88

Piguet P F et al., Evolution of collagen arthritis in mice is arrested by treatment with anti-tumour necrosis factor (TNF) antibody or recombinant soluble TNF receptor. 1992 Immunology, 77:510-14

Wooley P H et al, 1993, Influence of a recombinant human soluble tumour necrosis factor receptor Fc fusion protein on type II collagen-induced arthritis in mice. J. Immunology, 151:6602-7

Measured endpoints are arthritis index scores, diameter of swollen joints, numbers of swollen joints and the time to development of the above mentioned symptoms, as well as histological assessment of architecture of the joints.

### Assay (III)

Measurements of the affinities of the TNFR fragment can be measured in several assays. One being TNF induced cytotoxicity assay in WEHI 164 cells. The cells are cultured overnight in the absence of serum, in the presence of cyclohexamide and ED50 values of TNFα (3 nM) and a dilution curve of a TNFR fragment.

Alternatively, a scintillation proximity assay can be set up, using SPA beads with streptavidin coupled to a biotinylated TNFR, TNFR fragment or TNFR fusion protein. the beads are then incubated with ¹²⁵I-TNFα and a dilution curve of the TNFR fragment.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way,

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents,

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

1. A compound represented by the general formula 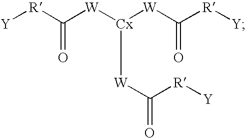
wherein Cx represents a center moiety, which is supporting three independent arms;
each W independently represents C₁₋₆-alkylene, C₁₋₆-alkyleneoxy, C₁₋₆-alkylene, C₂₋₆-alkenylene, C₂₋₆-alkynylene, hydroxy-C₁₋₆-alkylene, hydroxy-C₂₋₆-alkenylene, C₁₋₆-alkyleneoxy, C₂₋₆-alkenyleneoxy, C₂₋₆-alkanoylene, C₂₋₆-alkenoylene or a bond;
each R′ is the same or different, and comprises one or more of the group A and optionally one or more of the group B, each R′ starting with A,
wherein A is 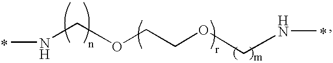
wherein r is any number from 1-50, and n and m are integers ≧2,
and wherein B is 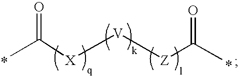
wherein X, V and Z are independently selected from —CR¹R²—, C₃₋₈— cycloalkylene, C₄₋₈-cycloalkenylene, -arylene-, -heteroarylene-, -heterocyclylene-, —O—, —S—, NR¹, —OCH₂CH₂O—, —OCH₂—, —CH₂O—;
R¹ and R² are independently selected from H, C₁₋₆-alkyl, C₂₋₆-alkenyl, C₃₋₈-cycloalkyl, C₄₋₈-cycloalkenyl, or R¹ and R² can be taken together to form a C₂₋₆-alkylene-bridge, and
q, k and l are independently selected from 0, 1, 2, 3, 4, 5 or 6, but they are not all simultaneously 0; and
Y is a group suitable for covalent attachment to a peptide.

2. A compound according to claim 1 which is represented by

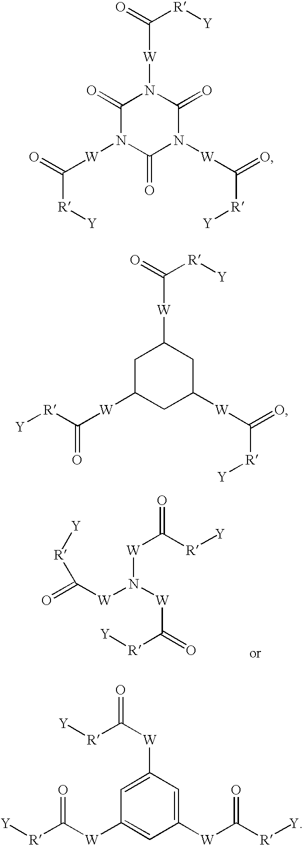

3. A compound according to claim 1 wherein W is a bond or C₁₋₆-alkylene.

4. A compound according to claim 3 represented by

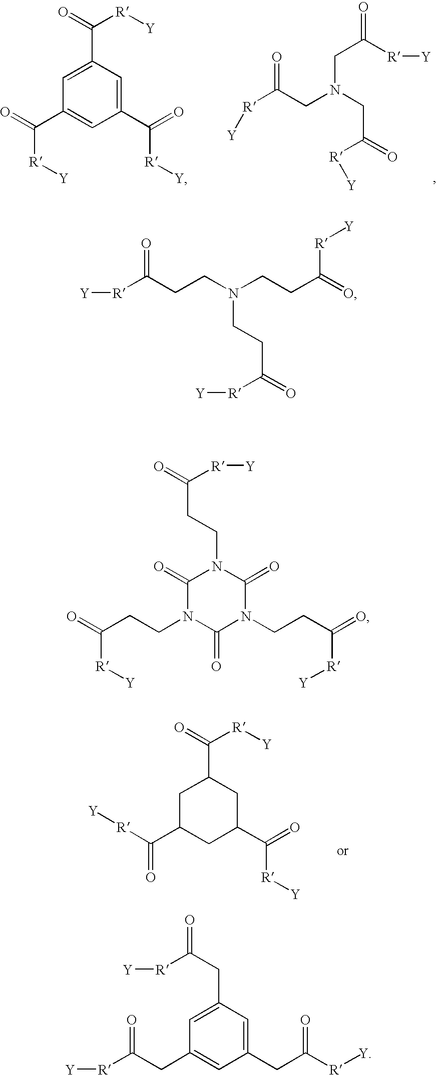

5. A compound according to claim 4, wherein A is 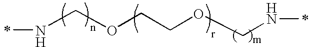
and r is 0-25.

6. The compound according to claim 5, wherein r is 0-10.

7. The compound according to claim 6, wherein r is 0-5.

8. The compound according to claim 7, wherein r is 0, 1, 2 or 3.

9. A compound according to claim 8 wherein A is

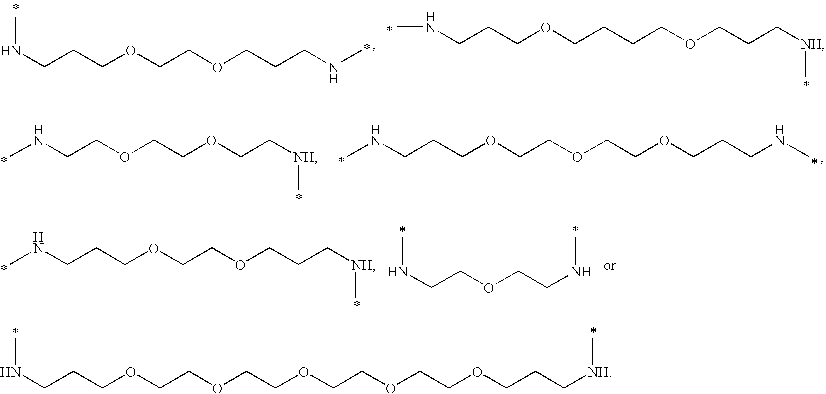

10. A compound according to claim 9, wherein X, V and Z independently represent —CR¹R²—, —O—, —S—, —NR¹—, —OCH₂CH₂O—, —OCH₂—, or —CH₂O—.

11. A compound according to claim 10, wherein X, V and Z independently represent —CR¹R²—.

12. A compound according to claim 11, wherein X, V and Z independently represent —OCH₂CH₂O—, —OCH₂—, or —CH₂O—.

13. A compound according to claim 10, wherein X, V and Z independently represent —(CH₂)₁₋₄—, —CH₂(OCH₂CH₂O)₁₋₆CH₂—, or —CH₂OCH₂—.

14. A compound according to claim 13, wherein X, V and Z independently represent —(CH₂)₃— or —CH₂OCH₂—.

15. A compound according to claim 14, wherein B is

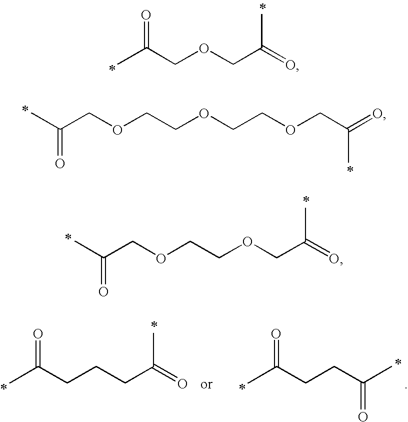

16. A compound according to claim 9, wherein X, V and Z independently represent arylene or heteroarylene.

17. A compound according to claim 16, wherein V is a phenylene.

18. A compound according to claim 9, wherein V is C₃₋₈-cycloalkylene or C₄₋₈-cycloalkenylene.

19. A compound according to claim 18, wherein V is cyclohexylene, cyclohexenylene or cyclopentylene.

20. A compound according to claim 9, wherein V is a di-radical derivative of morpholinyl, piperazinyl, dioxanyl or thienyl.

21. A compound according to claim 9, wherein Y is 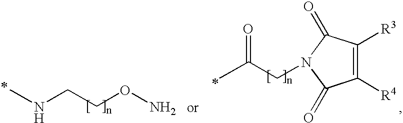
n is an integer ≧0, and
and R⁴ independently represent hydrogen, or C₁₋₆-alkyl.

22. A compound according to claim 21 wherein Y is

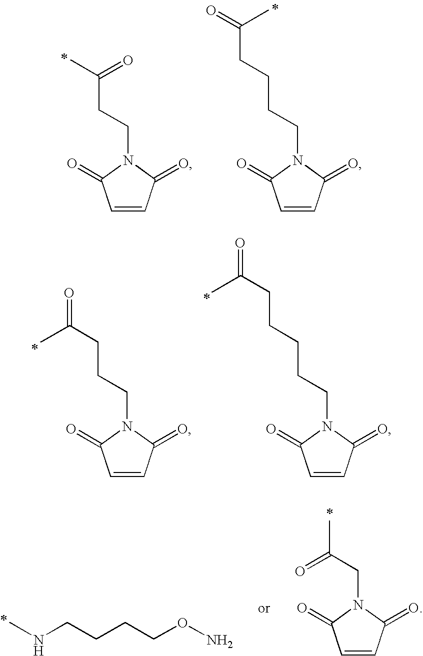

23. A compound according to claim 9, wherein Y is represented by

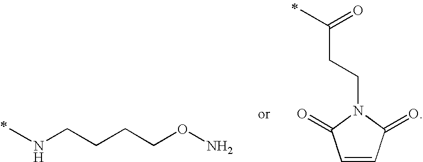

24. A compound according to claim 1, wherein R′ is selected from one of the following combinations: A-B, A-B-A-B, A-B-AB-A-B, A, A-B-A or A-B-A-B-A.

25. A compound according to claim 1, wherein two R′ are identical.

26. A compound according to claim 1, wherein all R′ are identical.

27. A compound according to claim 1, wherein all R′ are different.

28. A compound represented by the following formula:

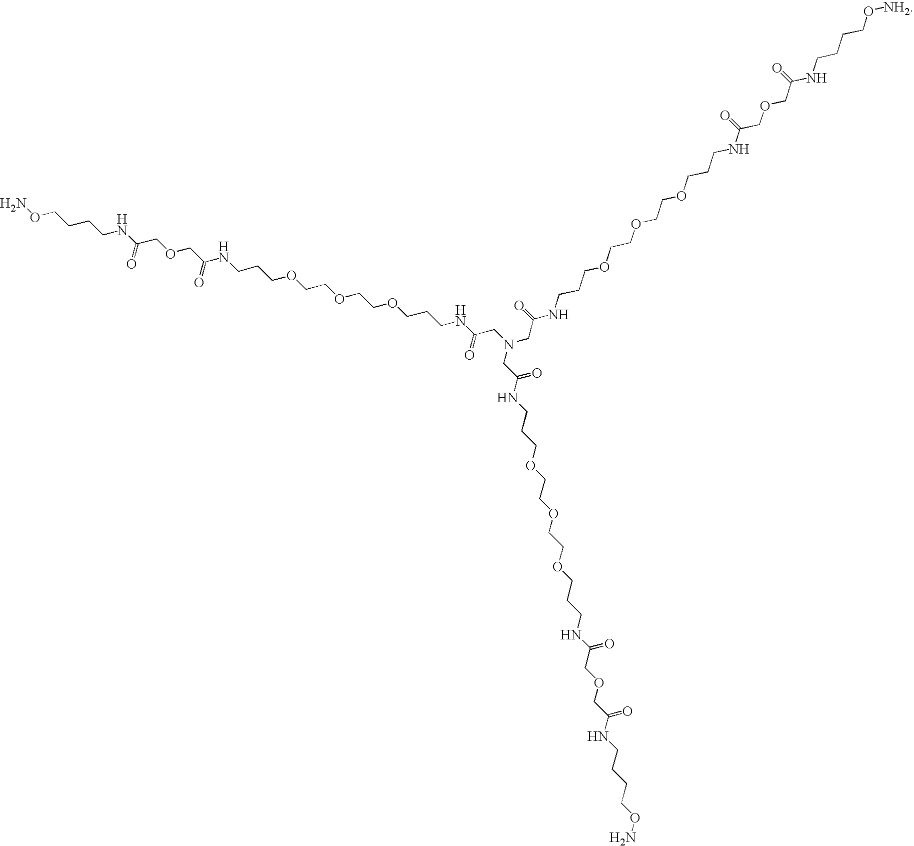

29. A peptide trimer comprising the compound according to claim 1, wherein the group Y is attached to a peptide P to form a group Y′P′—, the peptide trimer being represented by the general formula

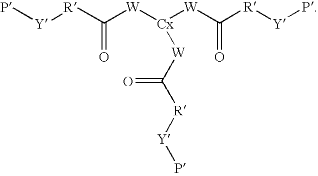

30. A peptide trimer according to claim 29, wherein P is a member of the TNFR superfamily.

31. A peptide trimer according to claim 29, wherein P is α-MSH, a fragment thereof, a functional analogue thereof, or a functional analogue of a fragment thereof.

32. A peptide trimer according to claim 29, wherein P is GLP-1, a fragment thereof, a functional analogue thereof, or a functional analogue of a fragment thereof.

33. A peptide trimer according to claim 29, wherein P is insulin, a fragment thereof, a functional analogue thereof, or a functional analogue of a fragment thereof.

34. A peptide trimer according to claim 29, wherein P is human growth hormone, a fragment thereof, a functional analogue thereof, or a functional analogue of a fragment thereof.

35. A peptide trimer according to claim 29, wherein P comprises the sequence of any one of SEQ ID NOS:1-7.

36. A peptide trimer according to claim 29, wherein P consists of the sequence of any one of SEQ ID NOS:1-7.

37. A polypeptide having the sequence of any of SEQ ID NOS:1-7.

38. A nucleic acid encoding a polypeptide according to claim 37.

39. A method of making a peptide trimer, comprising reacting a compound according to claim 1 with a peptide P to form a peptide trimer.

40. A method according to claim 39, wherein P is mutated or derivatised to introduce an amine, thiol, or hydroxyl group prior to the reacting.

41. A method according to claim 39, wherein P is selected from a member of the TNFR superfamily, α-MSH, GLP-1, insulin, human growth hormone, a fragment of any thereof, a functional analogue of any thereof, a functional analogue of a fragment of any thereof, and any combination thereof.

42. A method according to claim 41, wherein the functional analogue of the member of the TNFR superfamily is any one of SEQ ID NOS:1-7.

43. A method according to claim 39, wherein the compound is

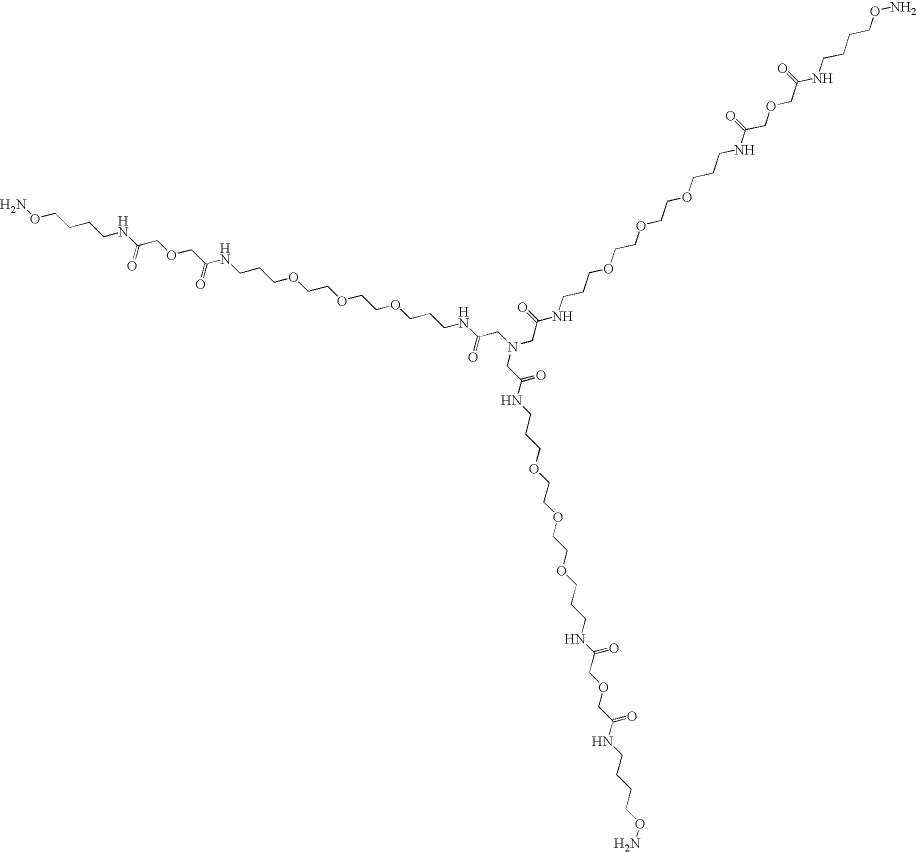

44. (canceled)

45. A pharmaceutical composition comprising a peptide trimer according to claim 29 together with a pharmaceutically acceptable excipient or carrier.

46. A compound according to claim 8 wherein A is

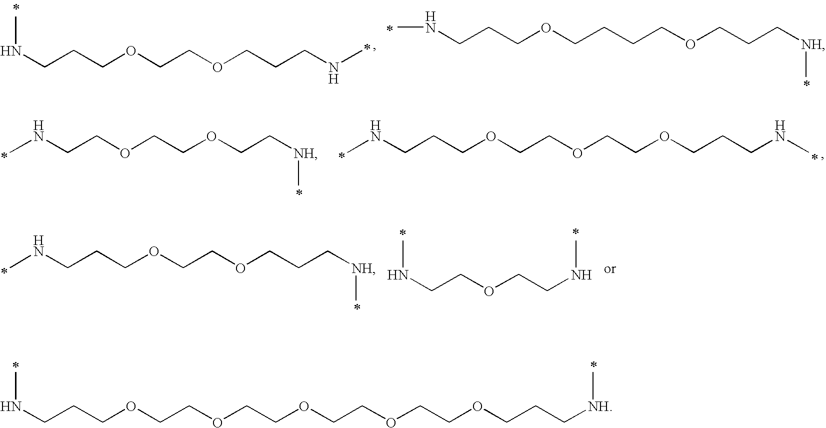

47. A compound according to claim 1, wherein X, V and Z independently represent —CR¹R²—, —O—, —S—, —NR¹—, —OCH₂CH₂O—, —OCH₂—, or —CH₂O—.

48. A compound according to claim 47, wherein X, V and Z independently represent —CR¹R²—.

49. A compound according to claim 48, wherein X, V and Z independently represent —OCH₂CH₂O—, —OCH₂—, or —CH₂O—.

50. A compound according to claim 47, wherein X, V and Z independently represent —(CH₂)₁₋₄—, —CH₂(OCH₂CH₂O)₁₋₆CH₂—, or —CH₂OCH₂—.

51. A compound according to claim 48, wherein X, V and Z independently represent —(CH₂)₁₋₄—, —CH₂(OCH₂CH₂O)₁₋₆CH₂—, or —CH₂OCH₂.

52. A compound according to claim 51, wherein X, V and Z independently represent —(CH₂)₃— or —CH₂OCH₂—.

53. A compound according to claim 47, wherein B is

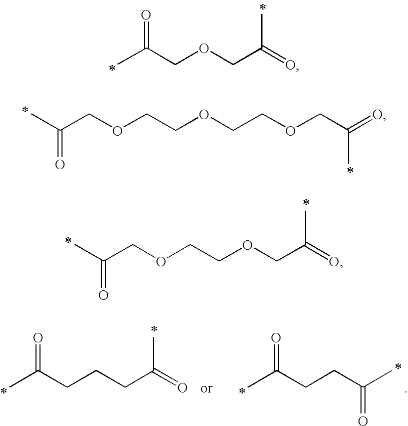

54. A compound according to claim 1, wherein X, V and Z independently represent arylene or heteroarylene.

55. A compound according to claim 54, wherein V is a phenylene.

56. A compound according to claim 1, wherein V is C₃₋₈-cycloalkylene or C₄₋₈-cycloalkenylene.

57. A compound according to claim 56, wherein V is cyclohexylene, cyclohexenylene or cyclopentylene.

58. A compound according to claim 1, wherein V is a di-radical derivative of morpholinyl, piperazinyl, dioxanyl or thienyl.

59. A compound according to claim 1, wherein Y is 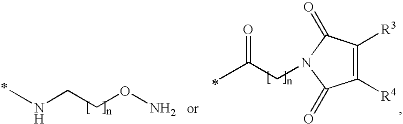
n is an integer ≧0, and
R³ and R⁴ independently represent hydrogen, or C₁₋₆-alkyl.

60. A compound according to claim 59 wherein Y is

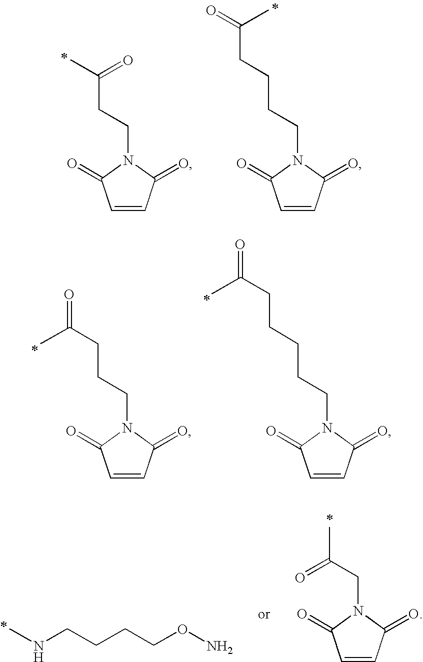

61. A compound according to claim 1, wherein Y is represented by

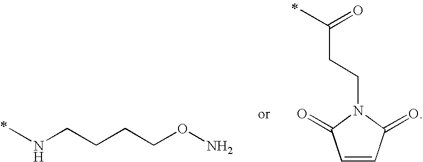

62. A compound according to claim 1 represented by

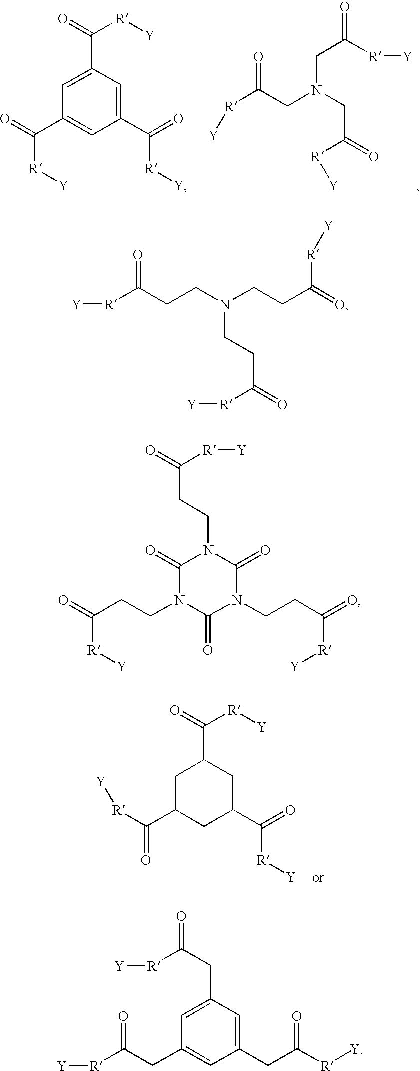

63. A compound according to claim 1, wherein A is 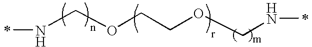
and r is 0-25.


SEQUENCE LISTING (17 sequences)

SEQ ID NO: 1 - protein, 131 aa, Artificial
  TNFR analogue
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTSHAA

SEQ ID NO: 2 - protein, 131 aa, Artificial
  TNFR analogue
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTSHAC

SEQ ID NO: 3 - protein, 129 aa, Artificial
  TNFR analogue
SVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTLLA

SEQ ID NO: 4 - protein, 132 aa, Artificial
  TNFR analogue
SVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTSHALLA

SEQ ID NO: 5 - protein, 125 aa, Artificial
  TNFR analogue
SVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVC

SEQ ID NO: 6 - protein, 130 aa, Artificial
  TNFR analogue
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTLLA

SEQ ID NO: 7 - protein, 133 aa, Artificial
  TNFR analogue
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTSHALLA

SEQ ID NO: 8 - protein, 171 aa, Homo sapiens
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 9 - protein, 161 aa, Homo sapiens
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIEN

SEQ ID NO: 10 - protein, 128 aa, Homo sapiens
DSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTC

SEQ ID NO: 11 - protein, 40 aa, Homo sapiens
MGLSTVPDLLLPLVLLELLVGIYPSGVIGLVPHLGDREKR

SEQ ID NO: 12 - DNA, 390 bp, Artificial
  TNFR analogue
gatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcaccaccagttgt

SEQ ID NO: 13 - DNA, 390 bp, Artificial
  TNFR analogue
gatagtgtgtgtccccaaggaaaatatatccaccctcaaaataattcgatttgctgtaccaagtgccacaaaggaacctacttgtacaatgactgtccaggcccggggcaggatacggactgcagggagtgtgagagcggctccttcaccgcttcagaaaaccacctcagacactgcctcagctgctccaaatgccgaaaggaaatgggtcaggtggagatctcttcttgcacagtggaccgggacaccgtgtgtggctgcaggaagaaccagtaccggcattattggagtgaaaaccttttccagtgcttcaattgcagcctctgcctcaatgggaccgtgcacctctcctgccaggagaaacagaacaccgtgtgcaccaccagtcac

SEQ ID NO: 14 - protein, 12 aa, Artificial
  MC4 peptide trimer (Example 3)
GSQHSXEXFRWK

SEQ ID NO: 15 - protein, 192 aa, Artificial
  Human growth hormone (hGH) with an N-terminally added serine residue
SFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEERIQTLMGRLEDGSPRTGQIFKQTYSKFDTNSHNDDALLKNYGLLYCFRKDMDKVETFLRIVQCRSVEGSCGF

SEQ ID NO: 16 - protein, 21 aa, Homo sapiens
GIVEQCCTSICSLYQLENYCN

SEQ ID NO: 17 - protein, 30 aa, Homo sapiens
FVNQHLCGSHLVEALYLVCGERGFFYTPKT